SEQUENCE LISTING (226 sequences)

SEQ ID NO: 1 - protein, 852 aa, Homo sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 2 - protein, 876 aa, Homo sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGQAGGLQDDSSGGYGDGQASGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQADTVKVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 3 - DNA, 30 bp, synthetic construct
  misc_feature 1..30: note = Description of Artificial Sequence: Synthetic primer
  source 1..30: mol_type = other DNA
gttcaagatctttatctgggaagaaggcgg

SEQ ID NO: 4 - DNA, 32 bp, synthetic construct
  misc_feature 1..32: note = Description of Artificial Sequence: Synthetic primer
  source 1..32: mol_type = other DNA
gaccttaattaaatgggcctcttcccttagaa

SEQ ID NO: 5 - protein, 9 aa, synthetic construct
  REGION 1..9: note = Description of Artificial Sequence: Synthetic peptide
GGSGGSGGS

SEQ ID NO: 6 - DNA, 17 bp, synthetic construct
  misc_feature 1..17: note = Description of Artificial Sequence: Synthetic oligonucleotide
  source 1..17: mol_type = other DNA
ttcagttgtggtatctg

SEQ ID NO: 7 - DNA, 17 bp, synthetic construct
  misc_feature 1..17: note = Description of Artificial Sequence: Synthetic oligonucleotide
  source 1..17: mol_type = other DNA
taccatgtctttctaag

SEQ ID NO: 8 - DNA, 20 bp, synthetic construct
  misc_feature 1..20: note = Description of Artificial Sequence: Synthetic oligonucleotide
  source 1..20: mol_type = other DNA
tttacagagctgtggtcact

SEQ ID NO: 9 - DNA, 18 bp, synthetic construct
  misc_feature 1..18: note = Description of Artificial Sequence: Synthetic oligonucleotide
  source 1..18: mol_type = other DNA
tccgtggctgccagtctg

SEQ ID NO: 10 - DNA, 18 bp, synthetic construct
  misc_feature 1..18: note = Description of Artificial Sequence: Synthetic oligonucleotide
  source 1..18: mol_type = other DNA
tgcatatactttttaatg

SEQ ID NO: 11 - DNA, 18 bp, synthetic construct
  misc_feature 1..18: note = Description of Artificial Sequence: Synthetic oligonucleotide
  source 1..18: mol_type = other DNA
tccaggagcgcgcctgag

SEQ ID NO: 12 - DNA, 20 bp, synthetic construct
  misc_feature 1..20: note = Description of Artificial Sequence: Synthetic oligonucleotide
  source 1..20: mol_type = other DNA
tgcctgtgaggaacagctgt

SEQ ID NO: 13 - DNA, 20 bp, synthetic construct
  misc_feature 1..20: note = Description of Artificial Sequence: Synthetic oligonucleotide
  source 1..20: mol_type = other DNA
tgcagacatctccaggctct

SEQ ID NO: 14 - DNA, 20 bp, synthetic construct
  misc_feature 1..20: note = Description of Artificial Sequence: Synthetic oligonucleotide
  source 1..20: mol_type = other DNA
taatttgtacatggttacat

SEQ ID NO: 15 - DNA, 20 bp, synthetic construct
  misc_feature 1..20: note = Description of Artificial Sequence: Synthetic oligonucleotide
  source 1..20: mol_type = other DNA
tgttagttaccatattgtgg

SEQ ID NO: 16 - DNA, 20 bp, synthetic construct
  misc_feature 1..20: note = Description of Artificial Sequence: Synthetic oligonucleotide
  source 1..20: mol_type = other DNA
tccagtccctggctcccatg

SEQ ID NO: 17 - DNA, 20 bp, synthetic construct
  misc_feature 1..20: note = Description of Artificial Sequence: Synthetic oligonucleotide
  source 1..20: mol_type = other DNA
tggctaatttttggtatttt

SEQ ID NO: 18 - DNA, 18 bp, synthetic construct
  misc_feature 1..18: note = Description of Artificial Sequence: Synthetic oligonucleotide
  source 1..18: mol_type = other DNA
tggctttccttccctttg

SEQ ID NO: 19 - DNA, 17 bp, synthetic construct
  misc_feature 1..17: note = Description of Artificial Sequence: Synthetic oligonucleotide
  source 1..17: mol_type = other DNA
tagccgcgaggaaggcg

SEQ ID NO: 20 - DNA, 20 bp, synthetic construct
  misc_feature 1..20: note = Description of Artificial Sequence: Synthetic oligonucleotide
  source 1..20: mol_type = other DNA
taaagacctgttacccaatt

SEQ ID NO: 21 - DNA, 20 bp, synthetic construct
  misc_feature 1..20: note = Description of Artificial Sequence: Synthetic oligonucleotide
  source 1..20: mol_type = other DNA
tcgtttttcttttttggaag

SEQ ID NO: 22 - DNA, 18 bp, synthetic construct
  misc_feature 1..18: note = Description of Artificial Sequence: Synthetic oligonucleotide
  source 1..18: mol_type = other DNA
ttctaaattgaggtgctg

SEQ ID NO: 23 - DNA, 20 bp, synthetic construct
  misc_feature 1..20: note = Description of Artificial Sequence: Synthetic oligonucleotide
  source 1..20: mol_type = other DNA
tcaatcattgcatgtttatt

SEQ ID NO: 24 - DNA, 18 bp, synthetic construct
  misc_feature 1..18: note = Description of Artificial Sequence: Synthetic oligonucleotide
  source 1..18: mol_type = other DNA
ttgcatctgggacagatg

SEQ ID NO: 25 - DNA, 17 bp, synthetic construct
  misc_feature 1..17: note = Description of Artificial Sequence: Synthetic oligonucleotide
  source 1..17: mol_type = other DNA
ttgatggtaacactatg

SEQ ID NO: 26 - DNA, 17 bp, synthetic construct
  misc_feature 1..17: note = Description of Artificial Sequence: Synthetic oligonucleotide
  source 1..17: mol_type = other DNA
ttatctccctcacccag

SEQ ID NO: 27 - DNA, 20 bp, synthetic construct
  misc_feature 1..20: note = Description of Artificial Sequence: Synthetic oligonucleotide
  source 1..20: mol_type = other DNA
tggttagaaacacagctgcc

SEQ ID NO: 28 - DNA, 20 bp, synthetic construct
  misc_feature 1..20: note = Description of Artificial Sequence: Synthetic oligonucleotide
  source 1..20: mol_type = other DNA
ttcatggttcaataaagact

SEQ ID NO: 29 - DNA, 18 bp, synthetic construct
  misc_feature 1..18: note = Description of Artificial Sequence: Synthetic oligonucleotide
  source 1..18: mol_type = other DNA
tacataaaatttttaagg

SEQ ID NO: 30 - DNA, 18 bp, synthetic construct
  misc_feature 1..18: note = Description of Artificial Sequence: Synthetic oligonucleotide
  source 1..18: mol_type = other DNA
ttaagcttctgaagtcag

SEQ ID NO: 31 - DNA, 18 bp, synthetic construct
  misc_feature 1..18: note = Description of Artificial Sequence: Synthetic oligonucleotide
  source 1..18: mol_type = other DNA
tgatcttcatttttaaag

SEQ ID NO: 32 - DNA, 18 bp, synthetic construct
  misc_feature 1..18: note = Description of Artificial Sequence: Synthetic oligonucleotide
  source 1..18: mol_type = other DNA
tggtatgagttgaaaatg

SEQ ID NO: 33 - DNA, 20 bp, synthetic construct
  misc_feature 1..20: note = Description of Artificial Sequence: Synthetic oligonucleotide
  source 1..20: mol_type = other DNA
taagtctacatatagtatcc

SEQ ID NO: 34 - DNA, 18 bp, synthetic construct
  misc_feature 1..18: note = Description of Artificial Sequence: Synthetic oligonucleotide
  source 1..18: mol_type = other DNA
taaaatgcactcacaatg

SEQ ID NO: 35 - DNA, 20 bp, synthetic construct
  misc_feature 1..20: note = Description of Artificial Sequence: Synthetic oligonucleotide
  source 1..20: mol_type = other DNA
tctctgaatcccctggtgac

SEQ ID NO: 36 - DNA, 17 bp, synthetic construct
  misc_feature 1..17: note = Description of Artificial Sequence: Synthetic oligonucleotide
  source 1..17: mol_type = other DNA
ttaaacagataagggag

SEQ ID NO: 37 - DNA, 18 bp, synthetic construct
  misc_feature 1..18: note = Description of Artificial Sequence: Synthetic oligonucleotide
  source 1..18: mol_type = other DNA
tggtgcgttatcagcctt

SEQ ID NO: 38 - DNA, 20 bp, synthetic construct
  misc_feature 1..20: note = Description of Artificial Sequence: Synthetic oligonucleotide
  source 1..20: mol_type = other DNA
tcaataccccacaaagaagc

SEQ ID NO: 39 - DNA, 18 bp, synthetic construct
  misc_feature 1..18: note = Description of Artificial Sequence: Synthetic oligonucleotide
  source 1..18: mol_type = other DNA
tctctaccttggaggctg

SEQ ID NO: 40 - DNA, 18 bp, synthetic construct
  misc_feature 1..18: note = Description of Artificial Sequence: Synthetic oligonucleotide
  source 1..18: mol_type = other DNA
tagaaaatacaacctcag

SEQ ID NO: 41 - DNA, 20 bp, synthetic construct
  misc_feature 1..20: note = Description of Artificial Sequence: Synthetic oligonucleotide
  source 1..20: mol_type = other DNA
tcctggaaaagccctctatg

SEQ ID NO: 42 - DNA, 20 bp, synthetic construct
  misc_feature 1..20: note = Description of Artificial Sequence: Synthetic oligonucleotide
  source 1..20: mol_type = other DNA
taagtttgcaaacaagctcc

SEQ ID NO: 43 - DNA, 20 bp, synthetic construct
  misc_feature 1..20: note = Description of Artificial Sequence: Synthetic oligonucleotide
  source 1..20: mol_type = other DNA
tggctttcctaggcagaagt

SEQ ID NO: 44 - DNA, 20 bp, synthetic construct
  misc_feature 1..20: note = Description of Artificial Sequence: Synthetic oligonucleotide
  source 1..20: mol_type = other DNA
tcacgcctttgtggccagag

SEQ ID NO: 45 - DNA, 20 bp, synthetic construct
  misc_feature 1..20: note = Description of Artificial Sequence: Synthetic oligonucleotide
  source 1..20: mol_type = other DNA
tcactgtgtacctttttatg

SEQ ID NO: 46 - DNA, 18 bp, synthetic construct
  misc_feature 1..18: note = Description of Artificial Sequence: Synthetic oligonucleotide
  source 1..18: mol_type = other DNA
tgcagtgcttcagccgct

SEQ ID NO: 47 - DNA, 20 bp, synthetic construct
  misc_feature 1..20: note = Description of Artificial Sequence: Synthetic primer
  source 1..20: mol_type = other DNA
ggaatcgtgaatacccctga

SEQ ID NO: 48 - DNA, 20 bp, synthetic construct
  misc_feature 1..20: note = Description of Artificial Sequence: Synthetic primer
  source 1..20: mol_type = other DNA
aacatgcaggtctgctttcc

SEQ ID NO: 49 - DNA, 20 bp, synthetic construct
  misc_feature 1..20: note = Description of Artificial Sequence: Synthetic primer
  source 1..20: mol_type = other DNA
ggaattggcctgcagaatta

SEQ ID NO: 50 - DNA, 20 bp, synthetic construct
  misc_feature 1..20: note = Description of Artificial Sequence: Synthetic primer
  source 1..20: mol_type = other DNA
gtacaccattggctggctct

SEQ ID NO: 51 - DNA, 20 bp, synthetic construct
  misc_feature 1..20: note = Description of Artificial Sequence: Synthetic primer
  source 1..20: mol_type = other DNA
tactgacccatgagcacagc

SEQ ID NO: 52 - DNA, 20 bp, synthetic construct
  misc_feature 1..20: note = Description of Artificial Sequence: Synthetic primer
  source 1..20: mol_type = other DNA
ccccactgccatcctactta

SEQ ID NO: 53 - DNA, 20 bp, synthetic construct
  misc_feature 1..20: note = Description of Artificial Sequence: Synthetic primer
  source 1..20: mol_type = other DNA
gagtgttggcagaatgagca

SEQ ID NO: 54 - DNA, 21 bp, synthetic construct
  misc_feature 1..21: note = Description of Artificial Sequence: Synthetic primer
  source 1..21: mol_type = other DNA
tgtgcgtatgcattttgttct

SEQ ID NO: 55 - DNA, 21 bp, synthetic construct
  misc_feature 1..21: note = Description of Artificial Sequence: Synthetic primer
  source 1..21: mol_type = other DNA
agcacacaattttgctcatca

SEQ ID NO: 56 - DNA, 20 bp, synthetic construct
  misc_feature 1..20: note = Description of Artificial Sequence: Synthetic primer
  source 1..20: mol_type = other DNA
acgtgcacatggaacaagac

SEQ ID NO: 57 - DNA, 20 bp, synthetic construct
  misc_feature 1..20: note = Description of Artificial Sequence: Synthetic primer
  source 1..20: mol_type = other DNA
ctgccaagtttctggttggt

SEQ ID NO: 58 - DNA, 20 bp, synthetic construct
  misc_feature 1..20: note = Description of Artificial Sequence: Synthetic primer
  source 1..20: mol_type = other DNA
gagacaaaatagcggggaca

SEQ ID NO: 59 - DNA, 20 bp, synthetic construct
  misc_feature 1..20: note = Description of Artificial Sequence: Synthetic primer
  source 1..20: mol_type = other DNA
aagaggacattctgggctga

SEQ ID NO: 60 - DNA, 20 bp, synthetic construct
  misc_feature 1..20: note = Description of Artificial Sequence: Synthetic primer
  source 1..20: mol_type = other DNA
cctgcctcctaagcttcctt

SEQ ID NO: 61 - DNA, 20 bp, synthetic construct
  misc_feature 1..20: note = Description of Artificial Sequence: Synthetic primer
  source 1..20: mol_type = other DNA
gacctgactcgaacccactc

SEQ ID NO: 62 - DNA, 20 bp, synthetic construct
  misc_feature 1..20: note = Description of Artificial Sequence: Synthetic primer
  source 1..20: mol_type = other DNA
gcctctgctaaggcacaaac

SEQ ID NO: 63 - DNA, 20 bp, synthetic construct
  misc_feature 1..20: note = Description of Artificial Sequence: Synthetic primer
  source 1..20: mol_type = other DNA
tgcctaggaaggcacttgtc

SEQ ID NO: 64 - DNA, 20 bp, synthetic construct
  misc_feature 1..20: note = Description of Artificial Sequence: Synthetic primer
  source 1..20: mol_type = other DNA
ggctggagatcagctttttg

SEQ ID NO: 65 - DNA, 20 bp, synthetic construct
  misc_feature 1..20: note = Description of Artificial Sequence: Synthetic primer
  source 1..20: mol_type = other DNA
tgtcctggaacggtttcact

SEQ ID NO: 66 - DNA, 20 bp, synthetic construct
  misc_feature 1..20: note = Description of Artificial Sequence: Synthetic primer
  source 1..20: mol_type = other DNA
tttctcctttgggcatcttg

SEQ ID NO: 67 - DNA, 20 bp, synthetic construct
  misc_feature 1..20: note = Description of Artificial Sequence: Synthetic primer
  source 1..20: mol_type = other DNA
aagaggacattctgggctga

SEQ ID NO: 68 - DNA, 20 bp, synthetic construct
  misc_feature 1..20: note = Description of Artificial Sequence: Synthetic primer
  source 1..20: mol_type = other DNA
cctgcctcctaagcttcctt

SEQ ID NO: 69 - DNA, 20 bp, synthetic construct
  misc_feature 1..20: note = Description of Artificial Sequence: Synthetic primer
  source 1..20: mol_type = other DNA
gacctgactcgaacccactc

SEQ ID NO: 70 - DNA, 20 bp, synthetic construct
  misc_feature 1..20: note = Description of Artificial Sequence: Synthetic primer
  source 1..20: mol_type = other DNA
gcctctgctaaggcacaaac

SEQ ID NO: 71 - DNA, 20 bp, synthetic construct
  misc_feature 1..20: note = Description of Artificial Sequence: Synthetic primer
  source 1..20: mol_type = other DNA
cccttgaccaggtaggttca

SEQ ID NO: 72 - DNA, 20 bp, synthetic construct
  misc_feature 1..20: note = Description of Artificial Sequence: Synthetic primer
  source 1..20: mol_type = other DNA
aaggagggctccagtttcat

SEQ ID NO: 73 - DNA, 21 bp, synthetic construct
  misc_feature 1..21: note = Description of Artificial Sequence: Synthetic primer
  source 1..21: mol_type = other DNA
tggtggaatgagtagcagagc

SEQ ID NO: 74 - DNA, 20 bp, synthetic construct
  misc_feature 1..20: note = Description of Artificial Sequence: Synthetic primer
  source 1..20: mol_type = other DNA
ggggattttcacacttggtg

SEQ ID NO: 75 - DNA, 20 bp, synthetic construct
  misc_feature 1..20: note = Description of Artificial Sequence: Synthetic primer
  source 1..20: mol_type = other DNA
tgtctgcacaaattgctgtg

SEQ ID NO: 76 - DNA, 20 bp, synthetic construct
  misc_feature 1..20: note = Description of Artificial Sequence: Synthetic primer
  source 1..20: mol_type = other DNA
cttgggaggggttcagagac

SEQ ID NO: 77 - DNA, 20 bp, synthetic construct
  misc_feature 1..20: note = Description of Artificial Sequence: Synthetic primer
  source 1..20: mol_type = other DNA
actcaaaggtgggtgtgagg

SEQ ID NO: 78 - DNA, 20 bp, synthetic construct
  misc_feature 1..20: note = Description of Artificial Sequence: Synthetic primer
  source 1..20: mol_type = other DNA
tccgataatctggtccaagg

SEQ ID NO: 79 - DNA, 21 bp, synthetic construct
  misc_feature 1..21: note = Description of Artificial Sequence: Synthetic primer
  source 1..21: mol_type = other DNA
cccaggaaacttgatgagaga

SEQ ID NO: 80 - DNA, 21 bp, synthetic construct
  misc_feature 1..21: note = Description of Artificial Sequence: Synthetic primer
  source 1..21: mol_type = other DNA
tgtggaaggagtgagtgaaca

SEQ ID NO: 81 - DNA, 21 bp, synthetic construct
  misc_feature 1..21: note = Description of Artificial Sequence: Synthetic primer
  source 1..21: mol_type = other DNA
gggttttcatgaagctttgaa

SEQ ID NO: 82 - DNA, 20 bp, synthetic construct
  misc_feature 1..20: note = Description of Artificial Sequence: Synthetic primer
  source 1..20: mol_type = other DNA
tttcgtattgcatcccatca

SEQ ID NO: 83 - DNA, 20 bp, synthetic construct
  misc_feature 1..20: note = Description of Artificial Sequence: Synthetic primer
  source 1..20: mol_type = other DNA
gctgagcttttcaggtaggc

SEQ ID NO: 84 - DNA, 20 bp, synthetic construct
  misc_feature 1..20: note = Description of Artificial Sequence: Synthetic primer
  source 1..20: mol_type = other DNA
gctcccaaaaagatgcaagt

SEQ ID NO: 85 - DNA, 20 bp, synthetic construct
  misc_feature 1..20: note = Description of Artificial Sequence: Synthetic primer
  source 1..20: mol_type = other DNA
gggccctccttatacttgga

SEQ ID NO: 86 - DNA, 20 bp, synthetic construct
  misc_feature 1..20: note = Description of Artificial Sequence: Synthetic primer
  source 1..20: mol_type = other DNA
tggactgggaggaacatagc

SEQ ID NO: 87 - DNA, 20 bp, synthetic construct
  misc_feature 1..20: note = Description of Artificial Sequence: Synthetic primer
  source 1..20: mol_type = other DNA
tgctacgtgcagcgtattct

SEQ ID NO: 88 - DNA, 20 bp, synthetic construct
  misc_feature 1..20: note = Description of Artificial Sequence: Synthetic primer
  source 1..20: mol_type = other DNA
tgcaacgctatttctcagga

SEQ ID NO: 89 - DNA, 20 bp, synthetic construct
  misc_feature 1..20: note = Description of Artificial Sequence: Synthetic primer
  source 1..20: mol_type = other DNA
agcattttcagcctcagtgg

SEQ ID NO: 90 - DNA, 20 bp, synthetic construct
  misc_feature 1..20: note = Description of Artificial Sequence: Synthetic primer
  source 1..20: mol_type = other DNA
ccttgtagcacctctgtcca

SEQ ID NO: 91 - DNA, 20 bp, synthetic construct
  misc_feature 1..20: note = Description of Artificial Sequence: Synthetic primer
  source 1..20: mol_type = other DNA
cagacttctggaacgcagtg

SEQ ID NO: 92 - DNA, 20 bp, synthetic construct
  misc_feature 1..20: note = Description of Artificial Sequence: Synthetic primer
  source 1..20: mol_type = other DNA
tgtgacaggccaagtctcag

SEQ ID NO: 93 - DNA, 20 bp, synthetic construct
  misc_feature 1..20: note = Description of Artificial Sequence: Synthetic primer
  source 1..20: mol_type = other DNA
ctgacggtttatgagcagca

SEQ ID NO: 94 - DNA, 20 bp, synthetic construct
  misc_feature 1..20: note = Description of Artificial Sequence: Synthetic primer
  source 1..20: mol_type = other DNA
gtttcccacagttccctgaa

SEQ ID NO: 95 - DNA, 23 bp, synthetic construct
  misc_feature 1..23: note = Description of Artificial Sequence: Synthetic primer
  source 1..23: mol_type = other DNA
tgaagtccacatgtttagctcct

SEQ ID NO: 96 - DNA, 21 bp, synthetic construct
  misc_feature 1..21: note = Description of Artificial Sequence: Synthetic primer
  source 1..21: mol_type = other DNA
tggaaggaatgtgattccact

SEQ ID NO: 97 - DNA, 20 bp, synthetic construct
  misc_feature 1..20: note = Description of Artificial Sequence: Synthetic primer
  source 1..20: mol_type = other DNA
ttcaacagcaaccaggaatg

SEQ ID NO: 98 - DNA, 25 bp, synthetic construct
  misc_feature 1..25: note = Description of Artificial Sequence: Synthetic primer
  source 1..25: mol_type = other DNA
aagctcaaaaagaaaaacttcaaca

SEQ ID NO: 99 - DNA, 20 bp, synthetic construct
  misc_feature 1..20: note = Description of Artificial Sequence: Synthetic primer
  source 1..20: mol_type = other DNA
ccattttccgtacatggtga

SEQ ID NO: 100 - DNA, 20 bp, synthetic construct
  misc_feature 1..20: note = Description of Artificial Sequence: Synthetic primer
  source 1..20: mol_type = other DNA
ctggctgtagggctctgttc

SEQ ID NO: 101 - DNA, 20 bp, synthetic construct
  misc_feature 1..20: note = Description of Artificial Sequence: Synthetic primer
  source 1..20: mol_type = other DNA
gacggggaaggaagaaagaa

SEQ ID NO: 102 - DNA, 18 bp, synthetic construct
  misc_feature 1..18: note = Description of Artificial Sequence: Synthetic primer
  source 1..18: mol_type = other DNA
tcccagctctcgcagctt

SEQ ID NO: 103 - DNA, 20 bp, synthetic construct
  misc_feature 1..20: note = Description of Artificial Sequence: Synthetic primer
  source 1..20: mol_type = other DNA
tacacaacagcacccacaca

SEQ ID NO: 104 - DNA, 21 bp, synthetic construct
  misc_feature 1..21: note = Description of Artificial Sequence: Synthetic primer
  source 1..21: mol_type = other DNA
ccccatttcagttctttctca

SEQ ID NO: 105 - DNA, 20 bp, synthetic construct
  misc_feature 1..20: note = Description of Artificial Sequence: Synthetic primer
  source 1..20: mol_type = other DNA
tcttctgggtttgttggcta

SEQ ID NO: 106 - DNA, 20 bp, synthetic construct
  misc_feature 1..20: note = Description of Artificial Sequence: Synthetic primer
  source 1..20: mol_type = other DNA
ggcaccatgtgaactctcct

SEQ ID NO: 107 - DNA, 21 bp, synthetic construct
  misc_feature 1..21: note = Description of Artificial Sequence: Synthetic primer
  source 1..21: mol_type = other DNA
tccaactcaatgccttttctg

SEQ ID NO: 108 - DNA, 20 bp, synthetic construct
  misc_feature 1..20: note = Description of Artificial Sequence: Synthetic primer
  source 1..20: mol_type = other DNA
cacaggcaagattcccattt

SEQ ID NO: 109 - DNA, 20 bp, synthetic construct
  misc_feature 1..20: note = Description of Artificial Sequence: Synthetic primer
  source 1..20: mol_type = other DNA
aatggctctggagaaaagca

SEQ ID NO: 110 - DNA, 20 bp, synthetic construct
  misc_feature 1..20: note = Description of Artificial Sequence: Synthetic primer
  source 1..20: mol_type = other DNA
gcatgccagtctgaagatga

SEQ ID NO: 111 - DNA, 21 bp, synthetic construct
  misc_feature 1..21: note = Description of Artificial Sequence: Synthetic primer
  source 1..21: mol_type = other DNA
tgtgaacctcgagaagtgtga

SEQ ID NO: 112 - DNA, 20 bp, synthetic construct
  misc_feature 1..20: note = Description of Artificial Sequence: Synthetic primer
  source 1..20: mol_type = other DNA
ttgttgaggtgtgcatgagg

SEQ ID NO: 113 - DNA, 19 bp, synthetic construct
  misc_feature 1..19: note = Description of Artificial Sequence: Synthetic primer
  source 1..19: mol_type = other DNA
gtcatgtccagcaggatgc

SEQ ID NO: 114 - DNA, 20 bp, synthetic construct
  misc_feature 1..20: note = Description of Artificial Sequence: Synthetic primer
  source 1..20: mol_type = other DNA
atgcagctgacccattgttt

SEQ ID NO: 115 - DNA, 20 bp, synthetic construct
  misc_feature 1..20: note = Description of Artificial Sequence: Synthetic primer
  source 1..20: mol_type = other DNA
acgatggaggacattggaag

SEQ ID NO: 116 - DNA, 20 bp, synthetic construct
  misc_feature 1..20: note = Description of Artificial Sequence: Synthetic primer
  source 1..20: mol_type = other DNA
tgaaggcttttcaggagctt

SEQ ID NO: 117 - DNA, 21 bp, synthetic construct
  misc_feature 1..21: note = Description of Artificial Sequence: Synthetic primer
  source 1..21: mol_type = other DNA
ctgcaaacaaggtctttggac

SEQ ID NO: 118 - DNA, 20 bp, synthetic construct
  misc_feature 1..20: note = Description of Artificial Sequence: Synthetic primer
  source 1..20: mol_type = other DNA
aggcagctacctggttaagg

SEQ ID NO: 119 - DNA, 20 bp, synthetic construct
  misc_feature 1..20: note = Description of Artificial Sequence: Synthetic primer
  source 1..20: mol_type = other DNA
gtgaccttggagacgttgct

SEQ ID NO: 120 - DNA, 20 bp, synthetic construct
  misc_feature 1..20: note = Description of Artificial Sequence: Synthetic primer
  source 1..20: mol_type = other DNA
agcctcttgaaccagagcag

SEQ ID NO: 121 - DNA, 23 bp, synthetic construct
  misc_feature 1..23: note = Description of Artificial Sequence: Synthetic primer
  source 1..23: mol_type = other DNA
aagagaaggagaaccaagcctta

SEQ ID NO: 122 - DNA, 20 bp, synthetic construct
  misc_feature 1..20: note = Description of Artificial Sequence: Synthetic primer
  source 1..20: mol_type = other DNA
cacaccagcaaagagcaaaa

SEQ ID NO: 123 - DNA, 20 bp, synthetic construct
  misc_feature 1..20: note = Description of Artificial Sequence: Synthetic primer
  source 1..20: mol_type = other DNA
gattccgggtcactgtgagt

SEQ ID NO: 124 - DNA, 20 bp, synthetic construct
  misc_feature 1..20: note = Description of Artificial Sequence: Synthetic primer
  source 1..20: mol_type = other DNA
ttttacggcgagatggtttc

SEQ ID NO: 125 - DNA, 22 bp, synthetic construct
  misc_feature 1..22: note = Description of Artificial Sequence: Synthetic primer
  source 1..22: mol_type = other DNA
ggaagaaaggaaggtaggaagg

SEQ ID NO: 126 - DNA, 20 bp, synthetic construct
  misc_feature 1..20: note = Description of Artificial Sequence: Synthetic primer
  source 1..20: mol_type = other DNA
agggcactctcctctcctct

SEQ ID NO: 127 - DNA, 20 bp, synthetic construct
  misc_feature 1..20: note = Description of Artificial Sequence: Synthetic primer
  source 1..20: mol_type = other DNA
gctgagaccacccactcttc

SEQ ID NO: 128 - DNA, 20 bp, synthetic construct
  misc_feature 1..20: note = Description of Artificial Sequence: Synthetic primer
  source 1..20: mol_type = other DNA
cccagaaggaattacccaca

SEQ ID NO: 129 - DNA, 20 bp, synthetic construct
  misc_feature 1..20: note = Description of Artificial Sequence: Synthetic primer
  source 1..20: mol_type = other DNA
tcacacatcacttgcgttca

SEQ ID NO: 130 - DNA, 20 bp, synthetic construct
  misc_feature 1..20: note = Description of Artificial Sequence: Synthetic primer
  source 1..20: mol_type = other DNA
tggcttgataacccaaccat

SEQ ID NO: 131 - DNA, 20 bp, synthetic construct
  misc_feature 1..20: note = Description of Artificial Sequence: Synthetic primer
  source 1..20: mol_type = other DNA
agggagcactctagggatgg

SEQ ID NO: 132 - DNA, 20 bp, synthetic construct
  misc_feature 1..20: note = Description of Artificial Sequence: Synthetic primer
  source 1..20: mol_type = other DNA
caggggaaacaggaagtgag

SEQ ID NO: 133 - DNA, 20 bp, synthetic construct
  misc_feature 1..20: note = Description of Artificial Sequence: Synthetic primer
  source 1..20: mol_type = other DNA
ccactaaaccgcaaccaaag

SEQ ID NO: 134 - DNA, 21 bp, synthetic construct
  misc_feature 1..21: note = Description of Artificial Sequence: Synthetic primer
  source 1..21: mol_type = other DNA
ggaaactcccagctttcaaac

SEQ ID NO: 135 - DNA, 20 bp, synthetic construct
  misc_feature 1..20: note = Description of Artificial Sequence: Synthetic primer
  source 1..20: mol_type = other DNA
cgtttctccctgggttcttt

SEQ ID NO: 136 - DNA, 20 bp, synthetic construct
  misc_feature 1..20: note = Description of Artificial Sequence: Synthetic primer
  source 1..20: mol_type = other DNA
atttttctgcctcccaaacc

SEQ ID NO: 137 - DNA, 20 bp, synthetic construct
  misc_feature 1..20: note = Description of Artificial Sequence: Synthetic primer
  source 1..20: mol_type = other DNA
ctgcccccaaagaaaggtat

SEQ ID NO: 138 - DNA, 21 bp, synthetic construct
  misc_feature 1..21: note = Description of Artificial Sequence: Synthetic primer
  source 1..21: mol_type = other DNA
ttggcatacttcatgctcaca

SEQ ID NO: 139 - DNA, 22 bp, synthetic construct
  misc_feature 1..22: note = Description of Artificial Sequence: Synthetic primer
  source 1..22: mol_type = other DNA
ttgacattaggtccaggtttga

SEQ ID NO: 140 - DNA, 20 bp, synthetic construct
  misc_feature 1..20: note = Description of Artificial Sequence: Synthetic primer
  source 1..20: mol_type = other DNA
tattttagggcaggcacacc

SEQ ID NO: 141 - DNA, 21 bp, synthetic construct
  misc_feature 1..21: note = Description of Artificial Sequence: Synthetic primer
  source 1..21: mol_type = other DNA
tcattttggtagcctttctgc

SEQ ID NO: 142 - DNA, 20 bp, synthetic construct
  misc_feature 1..20: note = Description of Artificial Sequence: Synthetic primer
  source 1..20: mol_type = other DNA
cactcaagtcccaggttggt

SEQ ID NO: 143 - DNA, 21 bp, synthetic construct
  misc_feature 1..21: note = Description of Artificial Sequence: Synthetic primer
  source 1..21: mol_type = other DNA
gatgatttggcttttgcgata

SEQ ID NO: 144 - DNA, 20 bp, synthetic construct
  misc_feature 1..20: note = Description of Artificial Sequence: Synthetic primer
  source 1..20: mol_type = other DNA
cttgtgggagctcgacatta

SEQ ID NO: 145 - DNA, 20 bp, synthetic construct
  misc_feature 1..20: note = Description of Artificial Sequence: Synthetic primer
  source 1..20: mol_type = other DNA
gacgtgttggtgcatacctg

SEQ ID NO: 146 - DNA, 20 bp, synthetic construct
  misc_feature 1..20: note = Description of Artificial Sequence: Synthetic primer
  source 1..20: mol_type = other DNA
atgaggctcctccctcattt

SEQ ID NO: 147 - DNA, 20 bp, synthetic construct
  misc_feature 1..20: note = Description of Artificial Sequence: Synthetic primer
  source 1..20: mol_type = other DNA
tcaagagtacggcaatcacg

SEQ ID NO: 148 - DNA, 20 bp, synthetic construct
  misc_feature 1..20: note = Description of Artificial Sequence: Synthetic primer
  source 1..20: mol_type = other DNA
gggaaaccgaaggattgatt

SEQ ID NO: 149 - DNA, 20 bp, synthetic construct
  misc_feature 1..20: note = Description of Artificial Sequence: Synthetic primer
  source 1..20: mol_type = other DNA
gaccaccggtcttctcatgt

SEQ ID NO: 150 - DNA, 20 bp, synthetic construct
  misc_feature 1..20: note = Description of Artificial Sequence: Synthetic primer
  source 1..20: mol_type = other DNA
gcagctgatgaagagcagaa

SEQ ID NO: 151 - DNA, 20 bp, synthetic construct
  misc_feature 1..20: note = Description of Artificial Sequence: Synthetic primer
  source 1..20: mol_type = other DNA
tagggtgtggatgtggaaca

SEQ ID NO: 152 - DNA, 20 bp, synthetic construct
  misc_feature 1..20: note = Description of Artificial Sequence: Synthetic primer
  source 1..20: mol_type = other DNA
tgggaaattgctgtgttgag

SEQ ID NO: 153 - DNA, 24 bp, synthetic construct
  misc_feature 1..24: note = Description of Artificial Sequence: Synthetic primer
  source 1..24: mol_type = other DNA
tcctgtaaagtcctcagatcaaca

SEQ ID NO: 154 - DNA, 20 bp, synthetic construct
  misc_feature 1..20: note = Description of Artificial Sequence: Synthetic primer
  source 1..20: mol_type = other DNA
gccagcttctaaggatgcac

SEQ ID NO: 155 - DNA, 20 bp, synthetic construct
  misc_feature 1..20: note = Description of Artificial Sequence: Synthetic primer
  source 1..20: mol_type = other DNA
ttggtctttggccttctagg

SEQ ID NO: 156 - DNA, 20 bp, synthetic construct
  misc_feature 1..20: note = Description of Artificial Sequence: Synthetic primer
  source 1..20: mol_type = other DNA
aatggggaagtgacaaggaa

SEQ ID NO: 157 - DNA, 22 bp, synthetic construct
  misc_feature 1..22: note = Description of Artificial Sequence: Synthetic primer
  source 1..22: mol_type = other DNA
cagcctttctaggaatcacaaa

SEQ ID NO: 158 - DNA, 21 bp, synthetic construct
  misc_feature 1..21: note = Description of Artificial Sequence: Synthetic primer
  source 1..21: mol_type = other DNA
ggatgatgaggaactggcttt

SEQ ID NO: 159 - DNA, 19 bp, synthetic construct
  misc_feature 1..19: note = Description of Artificial Sequence: Synthetic primer
  source 1..19: mol_type = other DNA
gtgaaccaccaagcacagc

SEQ ID NO: 160 - DNA, 20 bp, synthetic construct
  misc_feature 1..20: note = Description of Artificial Sequence: Synthetic primer
  source 1..20: mol_type = other DNA
agcaggggtggagagaaaat

SEQ ID NO: 161 - DNA, 20 bp, synthetic construct
  misc_feature 1..20: note = Description of Artificial Sequence: Synthetic primer
  source 1..20: mol_type = other DNA
ggctacagcgtcttcctgtg

SEQ ID NO: 162 - DNA, 20 bp, synthetic construct
  misc_feature 1..20: note = Description of Artificial Sequence: Synthetic primer
  source 1..20: mol_type = other DNA
cacacaccacacccacaact

SEQ ID NO: 163 - DNA, 20 bp, synthetic construct
  misc_feature 1..20: note = Description of Artificial Sequence: Synthetic primer
  source 1..20: mol_type = other DNA
taaggccggtctatcacagc

SEQ ID NO: 164 - DNA, 20 bp, synthetic construct
  misc_feature 1..20: note = Description of Artificial Sequence: Synthetic primer
  source 1..20: mol_type = other DNA
gcagtctcagcacctcaacc

SEQ ID NO: 165 - DNA, 20 bp, synthetic construct
  misc_feature 1..20: note = Description of Artificial Sequence: Synthetic primer
  source 1..20: mol_type = other DNA
actgcctgcctggagtctac

SEQ ID NO: 166 - DNA, 20 bp, synthetic construct
  misc_feature 1..20: note = Description of Artificial Sequence: Synthetic primer
  source 1..20: mol_type = other DNA
tcgctcactgaggaatgatg

SEQ ID NO: 167 - DNA, 20 bp, synthetic construct
  misc_feature 1..20: note = Description of Artificial Sequence: Synthetic primer
  source 1..20: mol_type = other DNA
tacaccgcgaagggatagtc

SEQ ID NO: 168 - DNA, 20 bp, synthetic construct
  misc_feature 1..20: note = Description of Artificial Sequence: Synthetic primer
  source 1..20: mol_type = other DNA
tgggggtcagagagagaatg

SEQ ID NO: 169 - DNA, 20 bp, synthetic construct
  misc_feature 1..20: note = Description of Artificial Sequence: Synthetic primer
  source 1..20: mol_type = other DNA
gggccccagactttaatttg

SEQ ID NO: 170 - DNA, 20 bp, synthetic construct
  misc_feature 1..20: note = Description of Artificial Sequence: Synthetic primer
  source 1..20: mol_type = other DNA
gcctctggagtgcagtacct

SEQ ID NO: 171 - DNA, 20 bp, synthetic construct
  misc_feature 1..20: note = Description of Artificial Sequence: Synthetic primer
  source 1..20: mol_type = other DNA
cccagatatttcctgctcca

SEQ ID NO: 172 - DNA, 20 bp, synthetic construct
  misc_feature 1..20: note = Description of Artificial Sequence: Synthetic primer
  source 1..20: mol_type = other DNA
cccccaaattccattattcc

SEQ ID NO: 173 - DNA, 19 bp, synthetic construct
  misc_feature 1..19: note = Description of Artificial Sequence: Synthetic primer
  source 1..19: mol_type = other DNA
gagggagcgagccatagtg

SEQ ID NO: 174 - DNA, 18 bp, synthetic construct
  misc_feature 1..18: note = Description of Artificial Sequence: Synthetic primer
  source 1..18: mol_type = other DNA
acaatggggctgcctgag

SEQ ID NO: 175 - DNA, 20 bp, synthetic construct
  misc_feature 1..20: note = Description of Artificial Sequence: Synthetic primer
  source 1..20: mol_type = other DNA
ggaggagggtggtctctcat

SEQ ID NO: 176 - DNA, 20 bp, synthetic construct
  misc_feature 1..20: note = Description of Artificial Sequence: Synthetic primer
  source 1..20: mol_type = other DNA
tcgaaagctacacggctctt

SEQ ID NO: 177 - DNA, 20 bp, synthetic construct
  misc_feature 1..20: note = Description of Artificial Sequence: Synthetic primer
  source 1..20: mol_type = other DNA
tgggtgaggaaggagaaaga

SEQ ID NO: 178 - DNA, 20 bp, synthetic construct
  misc_feature 1..20: note = Description of Artificial Sequence: Synthetic primer
  source 1..20: mol_type = other DNA
aaacccctatgggcaactct

SEQ ID NO: 179 - DNA, 20 bp, synthetic construct
  misc_feature 1..20: note = Description of Artificial Sequence: Synthetic primer
  source 1..20: mol_type = other DNA
ctggccctcttctcctttct

SEQ ID NO: 180 - DNA, 20 bp, synthetic construct
  misc_feature 1..20: note = Description of Artificial Sequence: Synthetic primer
  source 1..20: mol_type = other DNA
caatcatttgccaacacagg

SEQ ID NO: 181 - DNA, 20 bp, synthetic construct
  misc_feature 1..20: note = Description of Artificial Sequence: Synthetic primer
  source 1..20: mol_type = other DNA
gtctgaggaaaggcacctga

SEQ ID NO: 182 - DNA, 20 bp, synthetic construct
  misc_feature 1..20: note = Description of Artificial Sequence: Synthetic primer
  source 1..20: mol_type = other DNA
tcgcacctgtgtgagaggta

SEQ ID NO: 183 - DNA, 20 bp, synthetic construct
  misc_feature 1..20: note = Description of Artificial Sequence: Synthetic primer
  source 1..20: mol_type = other DNA
agcgacaaaaggtcaacaga

SEQ ID NO: 184 - DNA, 20 bp, synthetic construct
  misc_feature 1..20: note = Description of Artificial Sequence: Synthetic primer
  source 1..20: mol_type = other DNA
ggtgttgcggaaaacacttt

SEQ ID NO: 185 - DNA, 22 bp, synthetic construct
  misc_feature 1..22: note = Description of Artificial Sequence: Synthetic primer
  source 1..22: mol_type = other DNA
cctaagaatcagaaacgcaatg

SEQ ID NO: 186 - DNA, 20 bp, synthetic construct
  misc_feature 1..20: note = Description of Artificial Sequence: Synthetic primer
  source 1..20: mol_type = other DNA
cagtctgggcaacagaacaa

SEQ ID NO: 187 - DNA, 20 bp, synthetic construct
  misc_feature 1..20: note = Description of Artificial Sequence: Synthetic primer
  source 1..20: mol_type = other DNA
aacgaaacacaacctgcaca

SEQ ID NO: 188 - DNA, 20 bp, synthetic construct
  misc_feature 1..20: note = Description of Artificial Sequence: Synthetic primer
  source 1..20: mol_type = other DNA
ctgtaaccctacccccaacc

SEQ ID NO: 189 - DNA, 23 bp, synthetic construct
  misc_feature 1..23: note = Description of Artificial Sequence: Synthetic primer
  source 1..23: mol_type = other DNA
cagaacaaaatggagtcttagcc

SEQ ID NO: 190 - DNA, 20 bp, synthetic construct
  misc_feature 1..20: note = Description of Artificial Sequence: Synthetic primer
  source 1..20: mol_type = other DNA
tcagaaggtgtggggaaaag

SEQ ID NO: 191 - DNA, 20 bp, synthetic construct
  misc_feature 1..20: note = Description of Artificial Sequence: Synthetic primer
  source 1..20: mol_type = other DNA
atggctttcatgaagctgga

SEQ ID NO: 192 - DNA, 19 bp, synthetic construct
  misc_feature 1..19: note = Description of Artificial Sequence: Synthetic primer
  source 1..19: mol_type = other DNA
cgtctgtgcgaagagaagc

SEQ ID NO: 193 - DNA, 20 bp, synthetic construct
  misc_feature 1..20: note = Description of Artificial Sequence: Synthetic primer
  source 1..20: mol_type = other DNA
aaagcatttttgccatccag

SEQ ID NO: 194 - DNA, 20 bp, synthetic construct
  misc_feature 1..20: note = Description of Artificial Sequence: Synthetic primer
  source 1..20: mol_type = other DNA
ttcccggttagatgagttgg

SEQ ID NO: 195 - DNA, 20 bp, synthetic construct
  misc_feature 1..20: note = Description of Artificial Sequence: Synthetic primer
  source 1..20: mol_type = other DNA
gccctcccttgataagaacc

SEQ ID NO: 196 - DNA, 20 bp, synthetic construct
  misc_feature 1..20: note = Description of Artificial Sequence: Synthetic primer
  source 1..20: mol_type = other DNA
tgggaacctctccatctcac

SEQ ID NO: 197 - DNA, 21 bp, synthetic construct
  misc_feature 1..21: note = Description of Artificial Sequence: Synthetic primer
  source 1..21: mol_type = other DNA
ccaaagtcacatggatgacag

SEQ ID NO: 198 - DNA, 20 bp, synthetic construct
  misc_feature 1..20: note = Description of Artificial Sequence: Synthetic primer
  source 1..20: mol_type = other DNA
ggctaaatgaggcagatgct

SEQ ID NO: 199 - DNA, 20 bp, synthetic construct
  misc_feature 1..20: note = Description of Artificial Sequence: Synthetic primer
  source 1..20: mol_type = other DNA
ctgagggaagacatgctgga

SEQ ID NO: 200 - DNA, 20 bp, synthetic construct
  misc_feature 1..20: note = Description of Artificial Sequence: Synthetic primer
  source 1..20: mol_type = other DNA
agtgaagctccaccacctga

SEQ ID NO: 201 - DNA, 20 bp, synthetic construct
  misc_feature 1..20: note = Description of Artificial Sequence: Synthetic primer
  source 1..20: mol_type = other DNA
cccagctccacctggttatt

SEQ ID NO: 202 - DNA, 20 bp, synthetic construct
  misc_feature 1..20: note = Description of Artificial Sequence: Synthetic primer
  source 1..20: mol_type = other DNA
ctagggaagtgctgctgctg

SEQ ID NO: 203 - DNA, 20 bp, synthetic construct
  misc_feature 1..20: note = Description of Artificial Sequence: Synthetic primer
  source 1..20: mol_type = other DNA
atgtgtgacctgcacaccaa

SEQ ID NO: 204 - DNA, 20 bp, synthetic construct
  misc_feature 1..20: note = Description of Artificial Sequence: Synthetic primer
  source 1..20: mol_type = other DNA
ctttacccctgcgacgattt

SEQ ID NO: 205 - DNA, 20 bp, synthetic construct
  misc_feature 1..20: note = Description of Artificial Sequence: Synthetic primer
  source 1..20: mol_type = other DNA
ggcaccagcacagagttagc

SEQ ID NO: 206 - DNA, 20 bp, synthetic construct
  misc_feature 1..20: note = Description of Artificial Sequence: Synthetic primer
  source 1..20: mol_type = other DNA
gacgaccgaaggcactatca

SEQ ID NO: 207 - DNA, 20 bp, synthetic construct
  misc_feature 1..20: note = Description of Artificial Sequence: Synthetic primer
  source 1..20: mol_type = other DNA
gaagctcctccctcgtatgg

SEQ ID NO: 208 - DNA, 20 bp, synthetic construct
  misc_feature 1..20: note = Description of Artificial Sequence: Synthetic primer
  source 1..20: mol_type = other DNA
tccaagctgagatcgtaccg

SEQ ID NO: 209 - DNA, 20 bp, synthetic construct
  misc_feature 1..20: note = Description of Artificial Sequence: Synthetic primer
  source 1..20: mol_type = other DNA
atcagatttccagcctgtgc

SEQ ID NO: 210 - DNA, 20 bp, synthetic construct
  misc_feature 1..20: note = Description of Artificial Sequence: Synthetic primer
  source 1..20: mol_type = other DNA
tgatcacggaatcagcagtg

SEQ ID NO: 211 - DNA, 20 bp, synthetic construct
  misc_feature 1..20: note = Description of Artificial Sequence: Synthetic primer
  source 1..20: mol_type = other DNA
ctgggacagcaggaaaacag

SEQ ID NO: 212 - DNA, 20 bp, synthetic construct
  misc_feature 1..20: note = Description of Artificial Sequence: Synthetic primer
  source 1..20: mol_type = other DNA
tagattggaggggccacaag

SEQ ID NO: 213 - DNA, 20 bp, synthetic construct
  misc_feature 1..20: note = Description of Artificial Sequence: Synthetic primer
  source 1..20: mol_type = other DNA
ggcctgaaaatctgagctgc

SEQ ID NO: 214 - DNA, 21 bp, synthetic construct
  misc_feature 1..21: note = Description of Artificial Sequence: Synthetic primer
  source 1..21: mol_type = other DNA
cagtcgtctgtctcaactcca

SEQ ID NO: 215 - DNA, 20 bp, synthetic construct
  misc_feature 1..20: note = Description of Artificial Sequence: Synthetic primer
  source 1..20: mol_type = other DNA
gtacagcaaagggggtcagc

SEQ ID NO: 216 - DNA, 20 bp, synthetic construct
  misc_feature 1..20: note = Description of Artificial Sequence: Synthetic primer
  source 1..20: mol_type = other DNA
gactggcagcttgtagcctt

SEQ ID NO: 217 - DNA, 25 bp, synthetic construct
  misc_feature 1..25: note = Description of Artificial Sequence: Synthetic primer
  source 1..25: mol_type = other DNA
gttttatcttttgaaaggcacagtc

SEQ ID NO: 218 - DNA, 20 bp, synthetic construct
  misc_feature 1..20: note = Description of Artificial Sequence: Synthetic primer
  source 1..20: mol_type = other DNA
ttgtgatcaccaggtgcagt

SEQ ID NO: 219 - DNA, 20 bp, synthetic construct
  misc_feature 1..20: note = Description of Artificial Sequence: Synthetic primer
  source 1..20: mol_type = other DNA
tcaattcagctgcttcctca

SEQ ID NO: 220 - DNA, 20 bp, synthetic construct
  misc_feature 1..20: note = Description of Artificial Sequence: Synthetic primer
  source 1..20: mol_type = other DNA
ctggaaatctgatgggcact

SEQ ID NO: 221 - DNA, 20 bp, synthetic construct
  misc_feature 1..20: note = Description of Artificial Sequence: Synthetic primer
  source 1..20: mol_type = other DNA
tctgcactctggggaagaag

SEQ ID NO: 222 - DNA, 20 bp, synthetic construct
  misc_feature 1..20: note = Description of Artificial Sequence: Synthetic primer
  source 1..20: mol_type = other DNA
caagaatgaagcaagggaca

SEQ ID NO: 223 - DNA, 20 bp, synthetic construct
  misc_feature 1..20: note = Description of Artificial Sequence: Synthetic primer
  source 1..20: mol_type = other DNA
ttccccatgaaccacagttt

SEQ ID NO: 224 - DNA, 20 bp, synthetic construct
  misc_feature 1..20: note = Description of Artificial Sequence: Synthetic primer
  source 1..20: mol_type = other DNA
tgcaatactggagaggtgga

SEQ ID NO: 225 - DNA, 18 bp, synthetic construct
  misc_feature 1..18: note = Description of Artificial Sequence: Synthetic oligonucleotide
  source 1..18: mol_type = other DNA
ttaccatgtctttctaag

SEQ ID NO: 226 - protein, 9 aa, unidentified
  REGION 1..9: note = Description of Unknown: "LAGLIDADG" motif sequence
LAGLIDADG

(19) United States
(12) Patent Application Publication
(10) Pub. No.: US 2024/0254455 A1
(43) Pub. Date: Aug. 1, 2024

(54) TRANSCRIPTION ACTIVATOR-LIKE EFFECTOR (TALE) - LYSINE-SPECIFIC DEMETHYLASE 1 (LSD1) FUSION PROTEINS

(71) Applicant: The General Hospital Corporation, Boston, MA (US)

(72) Inventors: Jae Keith Joung, Winchester, MA (US); Eric M. Mendenhall, Madison, AL (US); Bradley E. Bernstein, Cambridge, MA (US); Deepak Reyon, Malden, MA (US)

(21) Appl. No.: 18/531,005

(22) Filed: Dec. 6, 2023

Related U.S. Application Data
(63) Continuation of application No. 16/376,736, filed on Apr. 5, 2019, now Pat. No. 11,891,631.
Application No. 16/376,736 is a continuation of application No. 14/435,065, filed on Apr. 10, 2015, now abandoned.
(60) Provisional application No. 61/865,432, filed on Aug. 13, 2013.
(60) Provisional application No. 61/776,039, filed on Mar. 11, 2013.
(60) Provisional application No. 61/713,098, filed on Oct. 12, 2012.

(51) Int. Cl. C12N 9/02; A61K 38/00; C07K 14/195; C12N 9/06; C12N 9/22; C12N 15/10; C12N 15/63

(52) U.S. Cl. CPC C12N 9/0004; C07K 14/195; C12N 9/0026; C12N 9/0083; C12N 9/22; C12Y 105/00; C12Y 114/99; A61K 38/00; C07K 2319/80; C12N 15/1093; C12N 15/63

(57) ABSTRACT

Fusion proteins comprising a DNA binding domain, e.g., a TAL effector repeat array (TALE) or zinc finger array, and a catalytic domain comprising a sequence that catalyzes histone demethylation, and methods of use thereof.

### CLAIM OF PRIORITY

This application is a continuation of U.S. patent application Ser. No. 16/376,736, filed Apr. 5, 2019, which is a continuation of U.S. patent application Ser. No. 14/435,065, filed Apr. 10, 2015, now Abandoned, which is a U.S. National Phase Application under 35 U.S.C. § 371 of International Patent Application No. PCT/US2013/064511, filed Oct. 11, 2013, which claims the benefit of, and incorporates by reference, U.S. Provisional Patent Applications Nos. 61/713,098, filed on Oct. 12, 2012: 61/776,039, filed on Mar. 11, 2013, and 61/865,432, filed on Aug. 13, 2013.

### FEDERALLY SPONSORED RESEARCH OR DEVELOPMENT

This invention was made with Government support under the National Human Genome Research Institute's ENCODE Project (Grant Nos. U54 HG004570, U54 HG006991) and Grant Nos. DP1 GM105378 and NIH P50 HG005550 awarded by the National Institutes of Health. The Government has certain rights in the invention.

### SEQUENCE LISTING

This application contains a Sequence Listing that has been submitted electronically as an XML file named “29539-0604003_SL_ST26.XML.” The XML file, created on Dec. 6, 2023, is 295,635 bytes in size. The material in the XML file is hereby incorporated by reference in its entirety.

### TECHNICAL FIELD

This invention relates to fusion proteins comprising a DNA binding domain, e.g., a TAL effector repeat array (TALE) or zinc finger, and a catalytic domain comprising a sequence that catalyzes histone demethylation, and methods of use thereof.

### BACKGROUND

Mammalian gene regulation is dependent on tissue-specific enhancers that can act across large distances to influence transcriptional activity¹⁻³. Mapping experiments have identified hundreds of thousands of putative enhancers whose functionality is supported by cell type-specific chromatin signatures and striking enrichments for disease-associated sequence variants⁴⁻¹¹. However, these studies do not address the in vivo functions of the putative elements or their chromatin states, and cannot determine which genes, if any, a given enhancer regulates.

### SUMMARY

The present invention is based, at least in part, on the development of fusions between transcription activator-like effector (TALE) repeat domains and a histone demethylase, e.g., Lysine-Specific Demethylase 1 (LSD1). As shown herein, these TALE-histone demethylase fusion proteins efficiently remove enhancer-associated chromatin modifications from target loci, without affecting control regions. Inactivation of enhancer chromatin by these fusions frequently causes down-regulation of proximal genes. These ‘epigenome editing’ tools can be used, e.g., to characterize a critical class of functional genomic elements, or to modulate (e.g., decrease) expression of selected genes).

Thus, provided herein are fusion proteins comprising an engineered DNA-binding domain that binds specifically to a preselected target sequence, and a catalytic domain comprising a sequence that catalyzes histone demethylation.

In another aspect, the invention provides methods for reducing methylation of histones associated with a selected DNA sequence in a mammalian cell. The methods include contacting the cell with a fusion protein comprising an engineered DNA-binding domain that binds specifically to a target sequence, wherein the target sequence is within about 10 kb, 5 kb, 2 kb, or 1 kb, 500 bp, 250 bp, 100 bp, 50 bp, 40 bp, 30 bp, or 20 bp, of the selected DNA sequence, and a catalytic domain comprising a sequence that catalyzes histone demethylation.

In another aspect, the invention provides methods for reducing methylation of histones associated with a selected DNA sequence in a mammalian cell. The methods include contacting the cell with a nucleic acid encoding a fusion protein comprising an engineered DNA-binding domain that binds specifically to a target sequence, wherein the target sequence is within about 10 kb, 5 kb, 2 kb, 1 kb, 500 bp, 250 bp, 100 bp, 50 bp, 40 bp, 30 bp, or 20 bp, of the selected DNA sequence, and a catalytic domain comprising a sequence that catalyzes histone demethylation.

In some embodiments, the fusion proteins comprise a linker between the DNA binding domain and the catalytic domain.

In some embodiments, the DNA-binding domain is or comprises an engineered transcription activator-like (TAL) effector repeat array, zinc finger, triplex-forming oligonucleotide, peptide nucleic acid, or a DNA-binding domain from a homing meganuclease (preferably a catalytically inactive homing meganuclease), or a catalytically inactive Cas9 nuclease.

In some embodiments, the catalytic domain comprises full length LSD1, or a catalytic domain of LSD1, e.g., amino acids 172-833 of the human LSD1 variant 2.

In some embodiments, the fusion proteins comprise a plurality of catalytic domains, optionally with linkers therebetween.

In some embodiments, the cell is a human cell.

In some embodiments, the cell is in a living mammal.

In some embodiments, the selected DNA sequence is a sequence of a p14ARF gene.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1F. Programmable TALE-LSD1 fusion modulates chromatin at an endogenous enhancer. (FIG. 1A) Schematic depicts workflow for identification of nucleosome-free target sequence (black stripe) within enhancer (peaks of histone modification) and design of corresponding TALE fusion. TALE arrays comprising ˜18 repeats (ovals) that each bind a single DNA base are fused to the LSD1 histone H3K4 demethylase. (TTACCATGTCTTTCTAAG, SEQ ID NO: 225) Upon transient transfection, we assayed for binding to the target site, induced chromatin changes and altered gene expression. (FIG. 1B) ChIP-seq signal tracks show H3K4me2, H3K27ac and TALE binding in K562 cells across a targeted enhancer in the SCL locus. Control tracks show anti-FLAG ChIP-seq signals in mCherry transfected cells and input chromatin. The target sequence of the TALE is indicated below. (FIG. 1C) ChIP-qPCR data show fold-change of H3K4me2 and H3K27ac enrichment in cells transfected with constructs encoding TALE-LSD1, the same TALE but lacking LSD1, or a ‘nontarget’ TALE-LSD1 whose cognate sequence is not present in the human genome. Data are presented as log 2 ratios normalized to mCherry plasmid transfected control (error bars represent+s.e.m. n=4 biological replicates). (FIG. 1D) ChIP-seq tracks show H3K4me2 and H3K27ac signals across the target SCL locus for K562 cells transfected with TALE-LSD1 or control mCherry plasmid. (FIG. 1E) ChIP-qPCR to test for off target effects of TALE-LSD1. ChIP-qPCR for H3K4me2 (lighter grey) and H3K27ac (darker grey) at two non-target control enhancers. For comparison, the data from the target enhancer is shown. (FIG. 1F) ChIP-qPCR values for the non-target control TALE-LSD1 (SEQ ID NO: 46). A TALE-LSD1 construct targeting a sequence not present in the human genome was transfected into K562 cells as a control for non-specific effects. Data is shown as ratio of enrichment to mCherry plasmid control for a subset of enhancers shown in FIG. 2. For comparison, an ‘on target’ TALE-LSD1 construct at its targeted enhancer is shown (TALE-LSD1 #4).

FIG. 2. TALE-LSD1 fusions targeting 40 candidate enhancers in K562 cells. The FLASH assembly method was used to engineer 40 TALE-LSD1 fusions that recognize 17−20 base sequences in nucleosome-free regions of candidate enhancers. These reagents were transfected into K562 cells and evaluated by ChIP-qPCR. Bi-directional plot shows fold change of H3K4me2 (lighter grey, left) and H3K27ac (darker grey, right) at the target locus for each of the 40 fusions, which are ordered by strength of effect and labeled by their target genomic site. Most target sites were evaluated using two qPCR primer sets. Data are presented as log 2 ratios normalized to mCherry plasmid transfected control (error bars represent +s.e.m., n=3 biological replicates). The solid lines (indicated at the bottom by arrows) define a 2-fold difference (log 2=−1). The horizontal dashed line demarcates constructs that induce a 2-fold reduction in histone modification levels for two or more of the four values shown. Regulated genes for 9 tested fusions are shown at right (see Examples and FIGS. 3A-3C). The data indicate that TALE-LSD1 reagents provide a general means for modulating chromatin state at endogenous enhancers.

FIGS. 3A-3C. TALE-LSD1 fusions to endogenous enhancers affect proximal gene expression. (FIG. 3A) Nine TALE-LSD1 fusions that robustly alter chromatin state (see FIG. 2) were evaluated for their effects on gene expression by RNA-seq (see Methods). For each of the nine fusions, a bar graph shows normalized gene expression values for the closest expressed upstream and downstream genes (error bars represent SEM, n=2 biological replicates). The light and dark grey bars (middle and right bars in each grouping) indicate the mean expression in cells transfected with the corresponding ‘on-target’ TALE-LSD1 construct, while the black bars (leftmost in each grouping) indicate the mean expression in cells transfected with control ‘off-target’ TALE constructs (error bars represent standard deviations, * indicates p<0.05). (FIG. 3B) ChIP-seq tracks show H3K4me2 and H3K27ac signals across the Zfpm2 locus. TALE-LSD1 fusions were designed to target candidate enhancers (black bars) in the first intron. (FIG. 3C) Bar graph shows relative ZFPM2 expression in K562 cells transfected with the indicated combinations of TALE-LSD1 constructs. Error bars indicate+s.e.m of 4 RT-qPCR measurements). The data suggest that these enhancers act redundantly in K562 cells to maintain ZFPM2 expression.

FIGS. 4A-4C. ChIP-qPCR to test for effects of TALE-LSD1. (FIG. 4A) ChIP-qPCR enrichment of H3K4me3 for three target enhancers, selected based on prior evidence of H4K4me3 (#4, #25) and one typical enhancer (#3) lacking K4me3. For comparision, data from a H3K4me3 enriched promoter is shown. (FIG. 4B) ChIP-qPCR for H3K4me3 (dark grey) at the two TALE-LSD1 targeted enhancers that showed some H3K4me3 enrichment. The data represent the decrease in enrichment at the target enhancer. (FIG. 4C) ChIP-qPCR enrichment of H3K4me1 for target enhancers of three TALE-LSD1 fusions. The data represent the decrease in enrichment at the target enhancer.

FIG. 5. ChIP-qPCR for H3K4me2 and H3K27ac at non-target sites. Data is shown for all 40 TALE-LSD1 constructs used in FIG. 2. Four primers sets were used to measure ChIP enrichment at two non-target enhancer loci for each TALE construct. No non-target enhancer showed a significant decrease (>2 fold decrease in 2/4 primer sets) in ChIP enrichment.

FIG. 6. ChIP-seq maps for H3K4me2 and H3K27ac for control cells and cells transfected independently with 2 TALE-LSD1 fusions.

FIG. 7. Mean normalized 3′ Digital Gene Expression Values for the 10-25 genes nearest the TALE target enhancer. Genes with values below 10 were considered unexpressed in K562 cells. Data points indicated with arrows and filled circles represent genes with a significant decrease in the TALE-LSD1 transfected cells. Significant decrease was considered if both biological replicates represented the two outlying values across all 22 RNA-seq datasets (see Methods).

FIGS. 8A-8B. Quantitative PCR confirmation of 3′ DGE. (FIG. 8A) RT-qPCR expression analysis for genes near two TALE-LSD1 target sites. (FIG. 8B) RT-qPCR data showing gene expression for Zfpm2 in cells transfected with a TALE #25 control plasmid that lacks the LSD1 protein, with data from the TALE-LSD1 for comparison. Error bars represent +SEM, n=2 biological replicates.

### DETAILED DESCRIPTION

Active enhancers are marked by histone H3 K4 mono- and di-methylation (H3K4me1 and H3K4me2) and K27 acetylation (H3K27ac)^{4.6.9.12.13}. The present inventors hypothesized that a given enhancer could be inactivated by removal of these chromatin marks. To test this hypothesis, monomeric fusions between TALE repeat arrays and the lysine-specific demethylase 1 (LSD1)¹⁴ were engineered. TALE repeats are modular DNA-binding domains that can be designed to bind essentially any genomic sequence of interest^{15.16}. LSD1 catalyzes the removal of H3 K4 and H3 K9 methylation^{1-3,14}. Although prior studies have used TALE nucleases to edit specific genomic regions to disrupt coding sequences^{4-11,17,18}, it was hypothesized that TALE-LSD1 fusions might provide a more versatile means for modulating the activity of noncoding elements and evaluating the significance of their chromatin states.

Described herein are fusion proteins comprising a DNA-binding domain (i.e., an engineered custom DNA-binding domain), and a catalytic domain (from a different protein) comprising a sequence that catalyzes histone demethylation (e.g., LSD1), with an optional linker between the two domains, such as a linker comprising 2-20, e.g., 10-12, amino acids, preferably a flexible linker (i.e., comprising amino acids such as Glycine and Serine that allow freedom in rotation). An exemplary linker comprises GGSGGSGGS (SEQ ID NO:5). Linkers are known in the art, see, Chen et al., e.g., Adv Drug Deliv Rev. 2012 Sep. 29. pii: S0169-409X(12)00300-6. As described herein, expression of a TAL effector repeat array-LSD1 (TAL-LSD1) fusion protein in human cells results in efficient removal of enhancer-associated chromatin modifications from target loci in close proximity to the target site bound by the TAL effector repeat array part of the protein.

Exemplified is a hybrid protein consisting of an engineered transcription activator-like (TAL) effector repeat array fused to a full length LSD1 protein, e.g., comprising the shorter variant 2 as set forth below; or a truncated form that retains the catalytic function of LSD1, e.g., as described herein. DNA-binding specificity is defined by the engineered TAL effector repeat array. These DNA-binding proteins can be engineered to bind to essentially any DNA sequence and published work from various labs, as well as the inventors' published and unpublished data, has demonstrated that these customizable domains can efficiently target a variety of fused domains to specific genomic locations (Reyon et al., FLASH assembly of TALENs for high-throughput genome editing. Nat Biotechnol (2012).doi:10.1038/nbt.2170: Moscou and Bogdanove, Science 326, 1501-1501 (2009): Boch et al., Science 326, 1509-1512 (2009): Miller et al., Nat Biotechnol 29, 143-148 (2010)). For example, engineered TAL effector repeat arrays have been fused to the cleavage domain of the FokI endonuclease as well as activators and repressors and act to target these domains to a user-defined sequence within the context of the genome.

### DNA-Binding Domains

The fusion proteins described herein can include any DNA Binding Domain (DBD) known in the art or engineered for a specific binding site. Exemplary DBDs include engineered or native TAL effector repeat arrays, engineered or native zinc fingers, modified variants (e.g., catalytically inactive) of homing meganucleases, modified variants (e.g., catalytically inactive) nucleases from the CRISPR-Cas system, chemical nucleases, and other native DBDs.

### TAL Effector Repeat Arrays

TAL effectors of plant pathogenic bacteria in the genus Xanthomonas play important roles in disease, or trigger defense, by binding host DNA and activating effector-specific host genes. Specificity depends on an effector-variable number of imperfect, typically ˜33-35 amino acid repeats. Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. In some embodiments, the polymorphic region that grants nucleotide specificity may be expressed as a triresidue or triplet.

Each DNA binding repeat can include a RVD that determines recognition of a base pair in the target DNA sequence, wherein each DNA binding repeat is responsible for recognizing one base pair in the target DNA sequence. In some embodiments, the RVD can comprise one or more of: HA for recognizing C: ND for recognizing C: HI for recognizing C: HN for recognizing G: NA for recognizing G: SN for recognizing G or A: YG for recognizing T: and NK for recognizing G and one or more of: HD for recognizing C: NG for recognizing T: NI for recognizing A: NN for recognizing G or A: NS for recognizing A or C or G or T: N* for recognizing C or T, wherein * represents a gap in the second position of the RVD; HG for recognizing T: H* for recognizing T, wherein * represents a gap in the second position of the RVD: and IG for recognizing T.

TALE proteins may be useful in research and biotechnology as targeted chimeric nucleases that can facilitate homologous recombination in genome engineering (e.g., to add or enhance traits useful for biofuels or biorenewables in plants). These proteins also may be useful as, for example, transcription factors, and especially for therapeutic applications requiring a very high level of specificity such as therapeutics against pathogens (e.g., viruses) as non-limiting examples.

Methods for generating engineered TALE arrays are known in the art, see, e.g., the fast ligation-based automatable solid-phase high-throughput (FLASH) system described in U.S. Ser. No. 61/610,212, and Reyon et al., Nature Biotechnology 30,460-465 (2012): as well as the methods described in Bogdanove & Voytas, Science 333, 1843-1846 (2011): Bogdanove et al., Curr Opin Plant Biol 13, 394-401 (2010): Scholze & Boch, J. Curr Opin Microbiol (2011): Boch et al., Science 326, 1509-1512 (2009): Moscou & Bogdanove, Science 326, 1501 (2009): Miller et al., Nat Biotechnol 29, 143-148 (2011); Morbitzer et al., T. Proc Natl Acad Sci USA 107, 21617-21622 (2010): Morbitzer et al., Nucleic Acids Res 39, 5790-5799 (2011): Zhang et al., Nat Biotechnol 29, 149-153 (2011): Geissler et al., PLOS ONE 6, e19509 (2011): Weber et al., PLOS ONE 6, e19722 (2011): Christian et al., Genetics 186, 757-761 (2010): Li et al., Nucleic Acids Res 39, 359-372 (2011): Mahfouz et al., Proc Natl Acad Sci USA 108, 2623-2628 (2011); Mussolino et al., Nucleic Acids Res (2011): Li et al., Nucleic Acids Res 39, 6315-6325 (2011): Cermak et al., Nucleic Acids Res 39, e82 (2011): Wood et al., Science 333, 307 (2011): Hockemeye et al. Nat Biotechnol 29, 731-734 (2011): Tesson et al., Nat Biotechnol 29, 695-696 (2011): Sander et al., Nat Biotechnol 29, 697-698 (2011): Huang et al., Nat Biotechnol 29, 699-700 (2011): and Zhang et al., Nat Biotechnol 29, 149-153 (2011): all of which are incorporated herein by reference in their entirety.

### Zinc Fingers

Zinc finger proteins are DNA-binding proteins that contain one or more zinc fingers, independently folded zinc-containing mini-domains, the structure of which is well known in the art and defined in, for example, Miller et al., 1985, EMBO J., 4:1609; Berg, 1988, Proc. Natl. Acad. Sci. USA, 85:99: Lee et al., 1989, Science. 245:635; and Klug, 1993, Gene, 135:83. Crystal structures of the zinc finger protein Zif268 and its variants bound to DNA show a semi-conserved pattern of interactions, in which typically three amino acids from the alpha-helix of the zinc finger contact three adjacent base pairs or a “subsite” in the DNA (Pavletich et al., 1991, Science, 252:809; Elrod-Erickson et al., 1998, Structure, 6:451). Thus, the crystal structure of Zif268 suggested that zinc finger DNA-binding domains might function in a modular manner with a one-to-one interaction between a zinc finger and a three-base-pair “subsite” in the DNA sequence. In naturally occurring zinc finger transcription factors, multiple zinc fingers are typically linked together in a tandem array to achieve sequence-specific recognition of a contiguous DNA sequence (Klug, 1993, Gene 135:83).

Multiple studies have shown that it is possible to artificially engineer the DNA binding characteristics of individual zinc fingers by randomizing the amino acids at the alpha-helical positions involved in DNA binding and using selection methodologies such as phage display to identify desired variants capable of binding to DNA target sites of interest (Rebar et al., 1994, Science, 263:671: Choo et al., 1994 Proc. Natl. Acad. Sci. USA, 91:11163: Jamieson et al., 1994, Biochemistry 33:5689: Wu et al., 1995 Proc. Natl. Acad. Sci. USA, 92: 344). Such recombinant zinc finger proteins can be fused to functional domains, such as transcriptional activators, transcriptional repressors, methylation domains, and nucleases to regulate gene expression, alter DNA methylation, and introduce targeted alterations into genomes of model organisms, plants, and human cells (Carroll, 2008, Gene Ther., 15:1463-68; Cathomen, 2008, Mol. Ther., 16:1200-07: Wu et al., 2007, Cell. Mol. Life Sci., 64:2933-44).

Widespread adoption and large-scale use of zinc finger protein technology have been hindered by the continued lack of a robust, easy-to-use, and publicly available method for engineering zinc finger arrays. One existing approach, known as “modular assembly,” advocates the simple joining together of pre-selected zinc finger modules into arrays (Segal et al., 2003, Biochemistry, 42:2137-48: Beerli et al., 2002, Nat. Biotechnol., 20:135-141: Mandell et al., 2006, Nucleic Acids Res., 34: W516-523; Carroll et al., 2006, Nat. Protoc. 1:1329-41: Liu et al., 2002, J. Biol. Chem., 277:3850-56; Bae et al., 2003, Nat. Biotechnol., 21:275-280; Wright et al., 2006, Nat. Protoc., 1:1637-52). Although straightforward enough to be practiced by any researcher, recent reports have demonstrated a high failure rate for this method, particularly in the context of zinc finger nucleases (Ramirez et al., 2008, Nat. Methods, 5:374-375: Kim et al., 2009, Genome Res. 19:1279-88), a limitation that typically necessitates the construction and cell-based testing of very large numbers of zinc finger proteins for any given target gene (Kim et al., 2009, Genome Res. 19:1279-88).

Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92: Isalan et al., 2001, Nat. Biotechnol., 19:656-660). In preferred embodiments, the zinc finger arrays are described in, or are generated as described in, WO 2011/017293 and WO 2004/099366. Additional suitable zinc finger DBDs are described in U.S. Pat. Nos. 6,511,808, 6,013,453, 6,007,988, and 6,503,717 and U.S. patent application 2002/0160940.

### Native DBDs

In some embodiments, a native DBD (e.g., a portion of a wild-type, non-engineered DNA binding protein that binds to a specific target sequence) can be used. For example, the DBD from a transcription factor, nuclease, histone, telomerase, or other DNA binding protein can be used. Typically DBDs include a structure that facilitates specific interaction with a target nucleic acid sequence: common DBD structures include helix-turn-helix: zinc finger: leucine zipper: winged helix: winged helix turn helix: helix-loop-helix: and hmg-box. The native DBD can be from any organism. See, e.g., Kummerfeld & Teichmann, Nucleic Acids Res. 34 (Database issue): D74-81 (2006). The residues in a DNA binding protein that contact DNA, and thus form part of the DBD, can be determined empirically or predicted computationally, e.g., as described in Tjong and Zhou, Nucl. Acids Res. 35:1465-1477 (2007). A database of DNA binding proteins can be used to identify DNA binding proteins and DBDs for use in the present compositions and methods: see, e.g., Harrison, Nature, 353, 715-719 (1991); Karmirantzou and Hamodrakas, Protein Eng. 14(7): 465-472 (2001); Kumar et al., BMC Bioinformatics. 8:463 (2007); Kumar et al., J Biomol Struct Dyn. 26(6):679-86 (2009): Lin et al., PLOS One. 6(9):e24756 (2011).

Where a native DBD is used in a fusion protein described herein, the catalytic domain is from a different protein.

### Homing Meganucleases

Meganucleases are sequence-specific endonucleases originating from a variety of organisms such as bacteria, yeast, algae and plant organelles. Endogenous meganucleases have recognition sites of 12 to 30 base pairs: customized DNA binding sites with 18 bp and 24 bp-long meganuclease recognition sites have been described, and either can be used in the present methods and constructs. See, e.g., Silva, G, et al., Current Gene Therapy, 11:11-27, (2011): Arnould et al., Journal of Molecular Biology, 355:443-58 (2006): Arnould et al., Protein Engineering Design & Selection, 24:27-31 (2011); and Stoddard, Q. Rev. Biophys. 38, 49 (2005): Grizot et al., Nucleic Acids Research, 38:2006-18 (2010). In some embodiments, catalytically inactive versions of the homing meganucleases are used, e.g., a mutant of I-Scel, e.g., comprising the mutation D44S, wherein the catalytically active aspartate from the first LAGLIDADG motif (SEQ ID NO: 226) is mutated to serine to make the enzyme inactive: N152K, reported to have ˜80% of the wt-activity: or the double variant D150C/N152K, which decreases the activity of the enzyme even further, e.g., as described in Gruen et al., Nucleic Acids Res. 2002:30:e29; Fonfara et al., Nucleic Acids Res. 2012 January: 40(2): 847-860; and Lippow et al., Nucleic Acids Res. 2009 May: 37(9):3061-73.

Nucleases from the CRISPR-Cas System

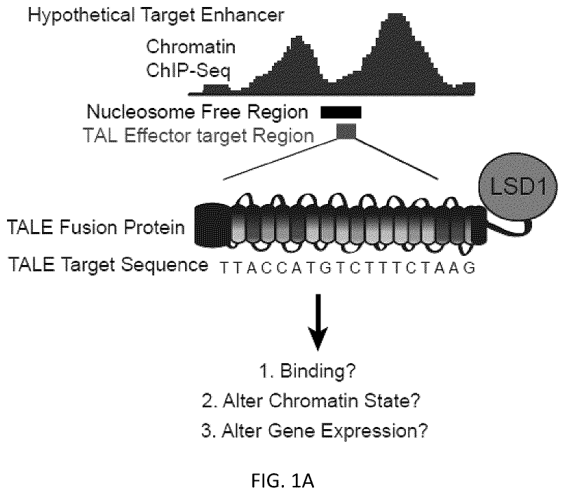

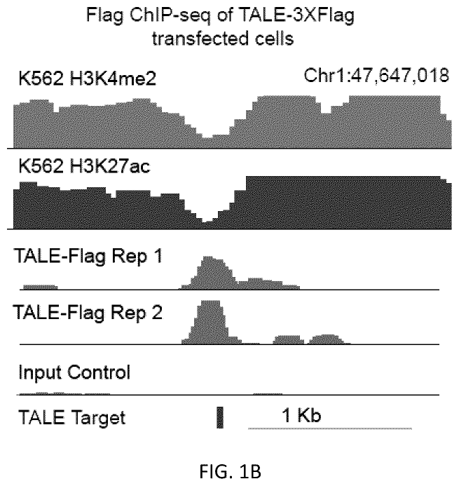

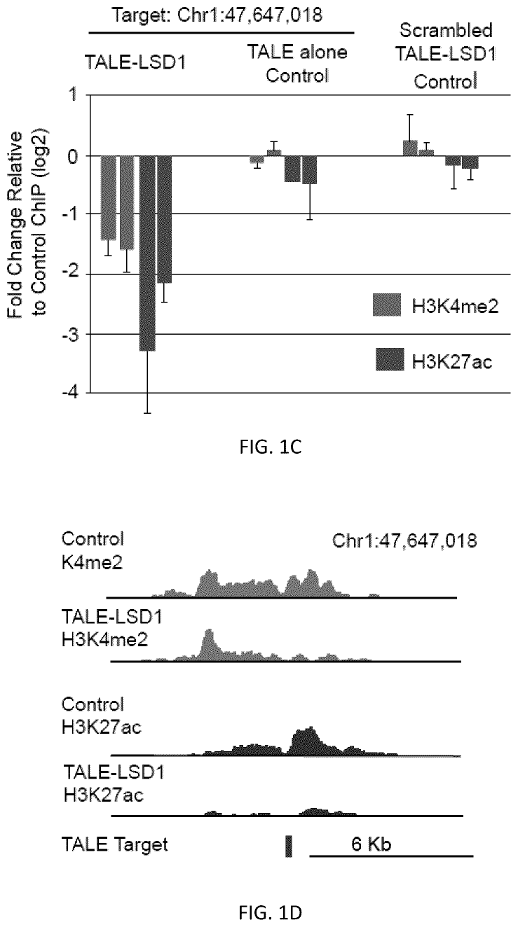

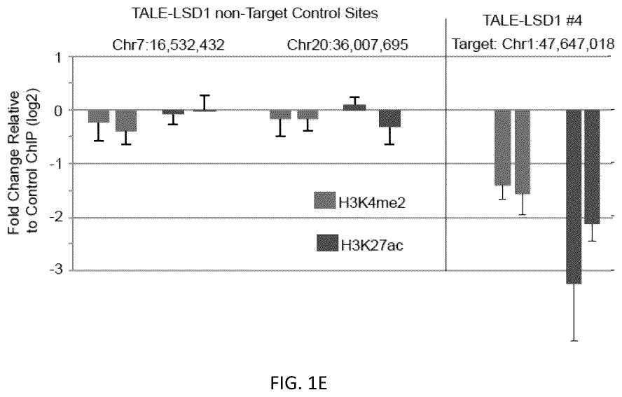

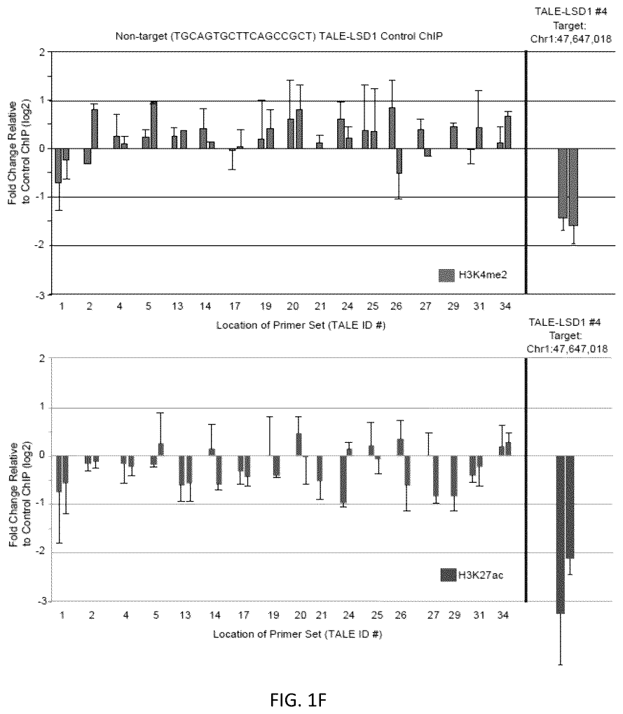

Catalytically inactive versions of the Cas9 nuclease can also be used as DBDs in the fusion proteins described herein: these fusion proteins are used in combination with a single guide RNA or a crRNA/tracrRNA pair for specificity. A number of bacteria express Cas9 protein variants. The Cas9 from Streptococcus pyogenes is presently the most commonly used: some of the other Cas9 proteins have high levels of sequence identity with the S. pyogenes Cas9 and use the same guide RNAs. Others are more diverse, use different gRNAs, and recognize different PAM sequences as well (the 2-5 nucleotide sequence specified by the protein which is adjacent to the sequence specified by the RNA). Chylinski et al. classified Cas9 proteins from a large group of bacteria (RNA Biology 10:5, 1-12: 2013), and a large number of Cas9 proteins are listed in supplementary FIG. 1 and supplementary table 1 thereof, which are incorporated by reference herein. The constructs and methods described herein can include the use of any of those Cas9 proteins, and their corresponding guide RNAs or other guide RNAs that are compatible. The Cas9 from Streptococcus thermophilus LMD-9 CRISPRI system has also been shown to function in human cells in Cong et al (Science 339, 819 (2013)). Additionally, Jinek et al. showed in vitro that Cas9 orthologs from S. thermophilus and L. innocua, (but not from N. meningitidis or C. jejuni, which likely use a different guide RNA), can be guided by a dual S. pyogenes gRNA to cleave target plasmid DNA, albeit with slightly decreased efficiency. These proteins are preferably mutated such that they retain their ability to be guided by the single guide RNA or a crRNA/tracrRNA pair and thus retain target specificity, but lack nuclease activity.

In some embodiments, the present system utilizes the Cas9 protein from S. pyogenes, either as encoded in bacteria or codon-optimized for expression in mammalian cells, containing D10A and H840A mutations to render the nuclease portion of the protein catalytically inactive: see, e.g., Jinek et al., Science 2012: 337:816-821: Qi et al., Cell 152, 1173-1183 (2013).

### Chemical Nucleases

DNA binding domains from the so-called “chemical nucleases,”(Pingoud and Silva, Nat Biotechnol. 25:743-4 (2007)), e.g., triplex-forming oligonucleotides or peptide nucleic acids can also be utilized in the present compositions and methods: see, e.g., Schleifman et al., Methods Mol Biol. 2008:435:175-90; Arimondo et al., Mol Cell Biol. 2006 Jan: 26(1):324-33: Majumdar et al., J Biol Chem. 2008 Apr 25: 283(17): 11244-52; Simon et al., Nucleic Acids Res. 2008 Jun: 36(11):3531-8: or Eisenschmidt et al., Nucleic Acids Res. 2005: 33(22): 7039-47.

### Catalytic Domains

The fusion proteins include a catalytic domain comprising a sequence that catalyzes histone demethylation. Exemplary proteins include the lysine (K)-specific demethylase 1A (KDM1A, also referred to herein as LSD1), a flavin adenine dinucleotide-dependent amino oxidase that catalyzes the removal of H3K4me1 and H3K4me2 (Shi et al., Cell 119:941-953 (2004); Metzger et al., Nature. 437(7057):436-9 (2005)).

Sequences for human LSD1 are known in the art and are shown in the following table:

| | GENBANK ® Accession Nos. | |
|---|---|---|
| Gene | Nucleic Acid | Amino Acid |
| LSD1-variant 1 | NM_001009999.2 (isoform a) | NP_001009999.1 |

| | |
|---|---|
| 1 | mlsgkkaaaa aaaaaaaatg teagpgtagg sengsevaaq paglsgpaev gpgavgertp |
| 61 | rkkepprasp pgglaeppgs agpqagptvv pgsatpmetg iaetpegrrt srrkrakvey |
| 121 | remdeslanl sedeyyseee rnakaekekk lpppppqapp eeenesepee psgqagglqd |
| 181 | dssggygdgq asgvegaafq srlphdrmts qeaacfpdii sgpqqtqkvf lfirnrtlql |
| 241 | wldnpkiqlt featlqqlea pynsdtvlvh rvhsylerhg linfgiykri kplptkktgk |
| 301 | viiigsgvsg laaarqlqsf gmdvtllear drvggrvatf rkgnyvadlg amvvtglggn |
| 361 | pmavvskqvn melakikqkc plyeangqad tvkvpkekde mveqefnrll eatsylshql |
| 421 | dfnvlnnkpv slgqalevvi qlqekhvkde qiehwkkivk tqeelkelln kmvnlkekik |
| 481 | elhqqykeas evkpprdita eflvkskhrd ltalckeyde laetqgklee klqeleanpp |
| 541 | sdvylssrdr qildwhfanl efanatplst lslkhwdqdd dfeftgshlt vrngyscvpv |
| 601 | alaegldikl ntavrqvryt asgceviavn trstsqtfiy kcdavlctlp lgvlkqqppa |
| 661 | vqfvpplpew ktsavqrmgf gnlnkvvlcf drvfwdpsvn lfghvgstta srgelflfwn |
| 721 | lykapillal vageaagime nisddvivgr clailkgifg ssavpqpket vvsrwradpw |
| 781 | argsysyvaa gssgndydlm aqpitpgpsi pgapqpiprl ffagehtirn ypatvhgall |
| 841 | sglreagria dqflgamytl prqatpgvpa qqspsm (SEQ ID NO: 2) |

| Gene | Nucleic Acid | Amino Acid |
|---|---|---|
| LSD1-variant 2* | NM_015013.3 (isoform b) | NP_055828.2 |

| | |
|---|---|
| 1 | mlsgkkaaaa aaaaaaaatg teagpgtagg sengsevaaq paglsgpaev gpgavgertp |
| 61 | rkkepprasp pgglaeppgs agpqagptvv pgsatpmetg iaetpegrrt srrkrakvey |
| 121 | remdeslanl sedeyyseee rnakaekekk lpppppqapp eeenesepee psgvegaafq |
| 181 | srlphdrmts qeaacfpdii sgpqqtqkvf lfirnrtlql wldnpkiqlt featlqqlea |
| 241 | pynsdtvlvh rvhsylerhg linfgiykri kplptkktgk viiigsgvsg laaarqlqsf |
| 301 | gmdvtllear drvggrvatf rkgnyvadlg amvvtglggn pmavvskqvn melakikqkc |
| 361 | plyeangqav pkekdemveq efnrlleats ylshqldfnv lnnkpvslgq alevviqlqe |
| 421 | khvkdeqieh wkkivktqee lkellnkmvn lkekikelhq qykeasevkp prditaeflv |
| 481 | kskhrdltal ckeydelaet qgkleeklqe leanppsdvy lssrdrqild whfanlefan |
| 541 | atplstlslk hwdqdddfef tgshltvrng yscvpvalae gldiklntav rqvrytasgc |
| 601 | eviavntrst sqtfiykcda vlctlplgvl kqqppavqfv pplpewktsa vqrmgfgnln |
| 661 | kvvlcfdrvf wdpsvnlfgh vgsttasrge lflfwnlyka pillalvage aagimenisd |
| 721 | dvivgrclai lkgifgssav pqpketvvsr wradpwargs ysyvaagssg ndydlmaqpi |
| 781 | tpgpsipgap qpiprlffag ehtirnypat vhgallsglr eagriadqfl gamytlprqa |
| 841 | tpgvpaqqsp sm (SEQ ID NO: 1) |

Variant 2, which was used in the exemplary fusion proteins described herein, lacks two alternate in-frame exons, compared to variant 1. The encoded protein (isoform b) is shorter than isoform a. LSD1 sequences from other species can also be used. See, e.g., FIG. 1 of Chen et al., PNAS Sep. 19, 2006 vol. 103 no. 38 13956-13961. In some embodiments, a fragment of LSD1 corresponding to residues 172-833 of the human LSD1 variant 2 (NP_055828.2) is used (Id.).

### Construction of Fusion Proteins

To generate a functional recombinant protein, the DNA binding domain is fused to at least one catalytic domain. Fusing catalytic domains to DBD to form functional fusion proteins involves only routine molecular biology techniques that are commonly practiced by those of skill in the art, see for example, U.S. Pat. Nos. 6,511,808, 6,013,453, 6,007,988, 6,503,717 and U.S. patent application 2002/0160940). Catalytic domains can be associated with the DBD domain at any suitable position, including the C- or N-terminus of the DBD.

In some embodiments, the fusion proteins can include multiple catalytic domains, e.g., on one or both ends of the DBD, e.g., concatenated together with an optional intervening linker: thus there can be one or more catalytic domains on each end of the DBD.

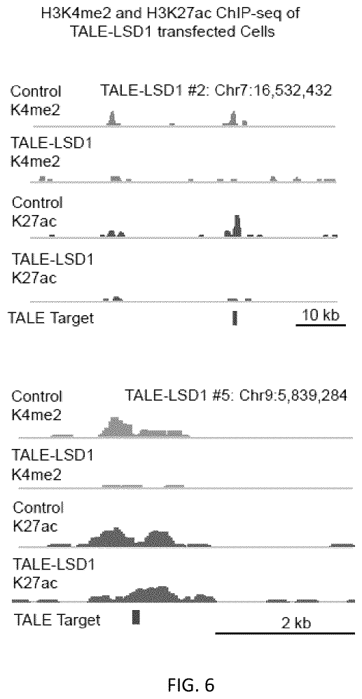

Alternatively, the catalytic domains, e.g., LSD1 units, could be multimerized through specific TALE DBD fused to concatenated protein-protein interaction domains (such as leucine zipper domains or ClonTech's iDimerize system, homodimerization and heterodimerization systems and ligands (e.g. AP20187, AP21967) which were previously provided by ARIAD under the brand name ARGENT. The B/B Homodimerizer (AP20187) induces dimerization of two proteins that each contain the DmrB homodimerization domain. The A/C Heterodimerizer (AP21967) induces dimerization of a protein possessing the DmrA domain and a second protein containing the DmrC domain. The D/D Solubilizer (alternative to AP21998) induces dissociation/disaggregation of proteins possessing DmrD domains. DmrD causes automatic self-association of proteins fused to it: see, e.g., Burnett et al., J. Leukoc. Biol. 75(4):612-623 (2004); Freeman et al., Cancer Res. 63(23):8256-8563 (2003): Castellano et al., Curr. Biol. 9(7): 351-360 (1999): Crabtree and Schreiber, Trends Biochem. Sci. 21(11): 418-422 (1996); Graef et al., Embo. J. 16(18): 5618-5628 (1997): Muthuswamy et al., Mol. Cell. Biol. 19(10): 6845-6857 (1999)). Thus, the catalytic domains fused to a DmrB, DmrA, or DmrD domains could be induced to interact with the TALE DBD in multiple copies. Alternatively, multimerization could be achieved through the use of split-inteins, a class of autocatyltic intein peptides that allow for the seamless covalent splicing of two separate proteins in a predictable and efficient manner (d′Avignon, et al., Biopolymers. 2006 Oct 15:83(3):255-67: Zitzewitz, et al., Biochemistry. 1995 Oct 3:34(39):12812-9; Li et al., Hum Gene Ther. 2008 Sep:19(9):958-64). Both the protein-protein interaction and intein approaches could be optimized to produce very long multimerized strings of catalytic domains. FIGS. 6A-D show exemplary schemes for multimerization.

### Methods of Use of the Fusion Proteins

The programmable DBD-LSD1 fusion proteins described herein can be used to modulate the chromatin state and regulatory activity of individual enhancers with high specificity. These reagents are generally useful for evaluating candidate enhancers identified in genomic mapping studies with higher throughput than direct genetic manipulations, particularly when combined with high-throughput methods for engineered TALE-based proteins²⁴. Moreover, the fusion proteins can be used to modulate (e.g., decrease) expression of developmental or disease-associated genes in specific contexts by inactivating their tissue-specific enhancers, and thus ultimately yield new therapeutic strategies. In some embodiments, the fusion proteins modulate the activity of an enhancer that only regulates a gene in a very specific context or cell type, rather than simply activating or repressing transcription by directly targeting a promoter. Unlike a promoter that would act in all tissues in which a gene is expressed, genes often have multiple enhancers that switch them on in different cell types or context. Thus the fusion proteins described herein can be designed to target enhancers that regulate the inappropriate expression (or repression) of a particular disease-associated gene in the disease context, and thereby correct the gene in that cell type (but leave it untouched in other cell types). For example, this could be used to regulate a gene that controls immune cell differentiation only in the correct immune cell type, and thus be a very specific way to alter the immune system and correct an autoimmune disorder. For example, BMP4 has tissue specific enhancers that regulate its expression in different tissues: see, e.g., Jumlongras et al., PLOS One. 2012:7(6):e38568. See also Ong and Corces, Nature Rev. Genetics 12:283-293 (2011). In some embodiments, the gene is described in Xie et al., Nature Genetics 45,836-841(2013): Gillies et al., Cell 33(3):717-728 (1983): Hoivik et al., Endocrinology. 2011 May:152(5):2100-12: Xu et al., Proc Natl Acad Sci USA. 104(30): 12377-12382 (2007).

The fusion proteins can be useful for the treatment of disease: for example, the fusion proteins can be targeted to a region of a gene that is overexpressed in a disease state, e.g., as a result of histone hypermethylation. See, e.g., Biancotto et al., Adv Genet. 2010:70:341-86 (cancer): Dreidax et al., Hum Mol Genet. 2013 May 1:22(9):1735-45) (p14ARF in neuroblastoma): Copeland et al., Oncogene. 2013 Feb 21:32(8):939-46 (cancer): Chase et al., Schizophr Res. 2013 Jun 28. pii: S0920-9964(13)00321-6 (schizophrenia); and Gavin et al., J Psychiatry Neurosci. 2009 May:34(3):232-7 (schizophrenia). Genes that are associated with hypermethylated histones can be identified using methods known in the art, e.g., chromatin immunoprecipitation (see, e.g., Dreidax et al., Hum Mol Genet. 2013 May 1:22(9):1735-45). In some embodiments, the methods include administering a fusion protein as described herein that comprises a DBD that targets p14ARF for the treatment of cancer, e.g., neuroblastoma.

In some embodiments, e.g., for the treatment of cancer or schizophrenia, a fusion protein as described herein that targets a gene that is underexpressed or overexpressed as a result of histone hypermethylation is administered, optionally in combination with a histone methyltransferase (HMT) inhibitor, e.g., BRD4770 (Methyl-2-benzamido-1-(3-phenylpropyl)-1H-benzo[d]imidazole-5-carboxylate): BIX 01294 (2-(Hexahydro-4-methyl-1H-1,4-diazepin-1-yl)-6,7-dimethoxy-N-[1-(phenylmethyl)-4-piperidinyl]-4-quinazolinamine trihydrochloride hydrate: Chaetocin (from Chaetomium minutum, PubChem Substance ID 24893002): or UNC0224 (7-[3-(dimethylamino)propoxy]-2-(hexahydro-4-methyl-1H-1,4-diazepin-1-yl)-6-methoxy-N-(1-methyl-4-piperidinyl)-4-quinazolinamine). See also Yost et al., Curr Chem Genomics. 2011:5(Suppl 1): 72-84.

The fusion proteins of the present invention are also useful as research tools: for example, in performing either in vivo or in vitro functional genomics studies (see, for example, U.S. Pat. No. 6,503,717, WO 2001019981, and U.S. patent publication 2002/0164575).

### Polypeptide Expression Systems

In order to use the fusion proteins described, it may be desirable to express the engineered proteins from a nucleic acid that encodes them. This can be performed in a variety of ways. For example, the nucleic acid encoding the fusion protein can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein or for production of the fusion protein. The nucleic acid encoding the fusion protein can also be cloned into an expression vector, for administration to a plant cell, animal cell, preferably a mammalian cell or a human cell, fungal cell, bacterial cell, or protozoan cell

To obtain expression, the fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990): and Current Protocols in Molecular Biology (Ausubel et al., eds., 2010). Bacterial expression systems for expressing the engineered TALE repeat protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of the fusion protein nucleic acid depends on the particular application. For example, a strong constitutive promoter is typically used for expression and purification of fusion proteins. In contrast, when the fusion protein is to be administered in vivo for gene regulation, either a constitutive or an inducible promoter can be used, depending on the particular use of the fusion protein. In addition, a preferred promoter for administration of the fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547: Oligino et al., 1998, Gene Ther., 5:491-496: Wang et al., 1997, Gene Ther., 4:432-441: Neering et al., 1996, Blood, 88: 1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. A preferred tag-fusion protein is the maltose binding protein, “MBP.” Such tag-fusion proteins can be used for purification of the engineered TALE repeat protein. Epitope tags can also be added to recombinant proteins to provide convenient methods of isolation, for monitoring expression, and for monitoring cellular and subcellular localization, e.g., c-myc or FLAG

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22: Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351: Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, poly brene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

In some embodiments, the fusion protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus. However, it may not be necessary to add a separate nuclear localization domain in cases where the DBD domain itself, or another functional domain within the final chimeric protein, has intrinsic nuclear translocation function.

### Use of Fusion Proteins in Gene Therapy

The fusion proteins described herein can be used to regulate gene expression or alter gene sequence in gene therapy applications. See for example U.S. Pat. Nos. 6,511,808, 6,013,453, 6,007,988, 6,503,717, U.S. patent application 2002/0164575, and U.S. patent application 2002/0160940. The methods can include administering nucleic acids encoding one or more of the fusion proteins described herein targeted to one or more genes. Since multiple histones across hundreds of basepairs of DNA in promoters or imprinted regions can influence gene expression, it may be desirable to reduce methylation of multiple histones, across longer sequences. If multiple histones, e.g., associated with a larger region of the genome (e.g., a large gene or gene cluster), are desired to be demethylated, a plurality of fusion proteins that target different positions on the same gene or general genomic region, e.g., targeting multiple positions tiled 1000, 500, 300, 250, 100, 50, or 20 bp of the central locus that will target each histone that is to be demethylated, can be administered. Alternatively or in addition, one or a plurality of fusion proteins that are multimerized as described herein can be administered.

Conventional viral and non-viral based gene transfer methods can be used to introduce nucleic acids encoding the fusion protein into mammalian cells or target tissues. Such methods can be used to administer nucleic acids encoding fusion proteins to cells in vitro. Preferably, the nucleic acids encoding the fusion proteins are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, 1992, Science, 256:808-813; Nabel & Felgner, 1993, TIBTECH, 11:211-217; Mitani & Caskey, 1993, TIBTECH, 11:162-166; Dillon, 1993, TIBTECH, 11:167-175; Miller, 1992, Nature, 357:455-460; Van Brunt, 1988, Biotechnology, 6:1149-54: Vigne, 1995, Restorat. Neurol. Neurosci., 8:35-36; Kremer & Perricaudet, 1995, Br. Med. Bull., 51:31-44: Haddada et al., in Current Topics in Microbiology and Immunology Doerfler and Bohm (eds) (1995): and Yu et al., 1994, Gene Ther., 1:13-26.

Methods of non-viral delivery of nucleic acids encoding the fusion proteins include lipofection, microinjection, biolistics, virosomes, liposomes, immunoliposomes, polycation or lipid:nucleic acid conjugates, naked DNA or RNA, artificial virions, and agent-enhanced uptake of DNA or RNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, 1995, Science, 270:404-410; Blaese et al., 1995, Cancer Gene Ther., 2:291-297; Behr et al., 1994, Bioconjugate Chem. 5:382-389; Remy et al., 1994, Bioconjugate Chem., 5:647-654; Gao et al., Gene Ther., 2:710-722; Ahmad et al., 1992, Cancer Res., 52:4817-20; U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding the fusion proteins takes advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of fusion proteins could include retroviral, lentivirus, adenoviral, adeno-associated, Sendai, and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992, J. Virol., 66:2731-39; Johann et al., 1992, J. Virol., 66:1635-40; Sommerfelt et al., 1990, Virololgy, 176:58-59: Wilson et al., 1989, J. Virol., 63:2374-78: Miller et al., 1991, J. Virol., 65:2220-24: WO 94/26877).

In applications where transient expression of the fusion protein is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987, Virology 160:38-47: U.S. Pat. No. 4,797,368: WO 93/24641: Kotin, 1994, Hum. Gene Ther., 5:793-801: Muzyczka, 1994, J. Clin. Invest., 94:1351). Construction of recombinant AAV vectors are described in a number of publications, including U.S. Pat. No. 5,173,414; Tratschin et al., 1985, Mol. Cell. Biol. 5:3251-60; Tratschin et al., 1984, Mol. Cell. Biol., 4:2072-81: Hermonat & Muzyczka, 1984, Proc. Natl. Acad. Sci. USA, 81:6466-70; and Samulski et al., 1989, J. Virol., 63:3822-28.

In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., 1995, Blood, 85:3048: Kohn et al., 1995, Nat. Med., 1: 1017; Malech et al., 1997, Proc. Natl. Acad. Sci. USA, 94:12133-38). PA317/pLASN was the first therapeutic vector used in a gene therapy trial. (Blaese et al., 1995, Science, 270:475-480). Transduction efficiencies of 50% or greater have been observed for MFG-S packaged vectors (Ellem et al., 1997, Immunol Immunother., 44: 10-20; Dranoffet al., 1997, Hum. Gene Ther., 1:111-112).

Recombinant adeno-associated virus vectors (rAAV) are a promising alternative gene delivery systems based on the defective and nonpathogenic parvovirus adeno-associated type 2 virus. Typically, the vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., 1998, Lancet, 351:1702-1703; Kearns et al., 1996, Gene Ther., 9:748-55).

Replication-deficient recombinant adenoviral vectors (Ad) are predominantly used for colon cancer gene therapy, because they can be produced at high titer and they readily infect a number of different cell types. Most adenovirus vectors are engineered such that a transgene replaces the Ad Ela, Elb, and E3 genes: subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., 1998, Hum. Gene Ther. 7:1083-89). Additional examples of the use of adenovirus vectors for gene transfer in clinical trials include Rosenecker et al., 1996, Infection, 24:15-10; Sterman et al., 1998, Hum. Gene Ther., 9:7 1083-89; Welsh et al., 1995, Hum. Gene Ther., 2:205-218: Alvarez et al., 1997, Hum. Gene Ther. 5:597-613: Topf et al., 1998, Gene Ther., 5:507-513: Sterman et al., 1998, Hum. Gene Ther., 7:1083-89.

Packaging cells are used to form virus particles that are capable of infecting a host cell. Such cells include 293 cells, which package adenovirus, and 4′2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, Proc. Natl. Acad. Sci. USA, 92:9747-51, reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below: Alternatively, vectors can be delivered to cells ex vivo, such as cells explanted from an individual patient (e.g., lymphocytes, bone marrow aspirates, tissue biopsy) or stem cells (e.g., universal donor hematopoietic stem cells, embryonic stem cells (ES), partially differentiated stem cells, non-pluripotent stem cells, pluripotent stem cells, induced pluripotent stem cells (iPS cells) (see e.g., Sipione et al., Diabetologia, 47:499-508, 2004)), followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with nucleic acid (gene or cDNA), encoding the fusion protein, and re-infused back into the subject organism (e.g., patient). Various cell types suitable for ex vivo transfection are well known to those of skill in the art (see, e.g., Freshney et al., Culture of Animal Cells, A Manual of Basic Technique (5th ed. 2005)) and the references cited therein for a discussion of how to isolate and culture cells from patients).

In one embodiment, stem cells (e.g., universal donor hematopoietic stem cells, embryonic stem cells (ES), partially differentiated stem cells, non-pluripotent stem cells, pluripotent stem cells, induced pluripotent stem cells (iPS cells) (see e.g., Sipione et al., Diabetologia. 47:499-508, 2004)) are used in ex vivo procedures for cell transfection and gene therapy. The advantage to using stem cells is that they can be differentiated into other cell types in vitro, or can be introduced into a mammal (such as the donor of the cells) where they will engraft in the bone marrow: Methods for differentiating CD34+ cells in vitro into clinically important immune cell types using cytokines such a GM-CSF, IFN-gamma and TNF-alpha are known (see Inaba et al., 1992, J. Exp. Med., 176:1693-1702).

Stem cells can be isolated for transduction and differentiation using known methods. For example, stem cells can be isolated from bone marrow cells by panning the bone marrow cells with antibodies which bind unwanted cells, such as CD4+ and CD8+(T cells), CD45+(panB cells), GR-1 (granulocytes), and lad (differentiated antigen presenting cells) (see Inaba et al., 1992, J. Exp. Med., 176:1693-1702).

Vectors (e.g., retroviruses, adenoviruses, liposomes, etc.) containing nucleic acids encoding the fusion protein can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Alternatively, stable formulations of the fusion protein can also be administered.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington: The Science and Practice of Pharmacy, 21st ed., 2005).

### Delivery Vehicles

An important factor in the administration of polypeptide compounds, such as the fusion proteins of the present invention, is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as fusion protein across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, 1996, Curr. Opin. Neurobiol., 6:629-634). Another subsequence, the h (hydrophobic) domain of signal peptides, was found to have similar cell membrane translocation characteristics (see, e.g., Lin et al., 1995, J. Biol. Chem., 270:14255-58).

Examples of peptide sequences that can be linked to a protein, for facilitating uptake of the protein into cells, include, but are not limited to: peptide fragments of the tat protein of HIV (Endoh et al., 2010, Methods Mol. Biol., 623:271-281: Schmidt et al., 2010, FEBS Lett., 584:1806-13: Futaki, 2006, Biopolymers, 84:241-249): a 20 residue peptide sequence which corresponds to amino acids 84-103 of the p16 protein (see Fahraeus et al., 1996, Curr. Biol., 6:84); the third helix of the 60-amino acid long homeodomain of Antennapedia (Derossi et al., 1994, J. Biol. Chem., 269: 10444): the h region of a signal peptide, such as the Kaposi fibroblast growth factor (K-FGF) h region (Lin et al., supra): the VP22 translocation domain from HSV (Elliot & O'Hare, 1997, Cell, 88:223-233): or supercharged proteins or intraphilins, e.g., as described in US20120100569; US20110112040; Thompson et al.. Methods in Enzymology, 503:293-319 (2012): Cronican et al (2011) Chem Biol. 18, 833: Cronican et al (2010) ACS Chem. Biol. 5, 747: McNaughton et al (2009) Proc. Natl. Acad. Sci. USA 106, 6111: and Lawrence et al (2007) J. Am. Chem. Soc. 129, 10110. See also, e.g., Caron et al., 2001, Mol Ther., 3:310-318: Langel, Cell-Penetrating Peptides: Processes and Applications (CRC Press, Boca Raton FL 2002): El-Andaloussi et al., 2005, Curr. Pharm. Des., 11:3597-3611: and Deshayes et al., 2005, Cell. Mol. Life Sci., 62:1839-49. Other suitable chemical moieties that provide enhanced cellular uptake may also be chemically linked to the Fusion proteins described herein.

Toxin molecules also have the ability to transport polypeptides across cell membranes. Often, such molecules are composed of at least two parts (called “binary toxins”): a translocation or binding domain or polypeptide and a separate toxin domain or polypeptide. Typically, the translocation domain or polypeptide binds to a cellular receptor. and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions (Arora et al., 1993, J. Biol. Chem., 268:3334-41; Perelle et al., 1993, Infect. Immun., 61:5147-56; Stenmark et al., 1991, J. Cell Biol., 113:1025-32; Donnelly et al., 1993, Proc. Natl. Acad. Sci. USA, 90:3530-34: Carbonetti et al., 1995, Abstr. Annu. Meet. Am. Soc. Microbiol. 95:295: Sebo et al., 1995, Infect. Immun., 63:3851-57; Klimpel et al., 1992, Proc. Natl. Acad. Sci. USA, 89:10277-81; and Novak et al., 1992, J. Biol. Chem., 267:17186-93).

Such subsequences can be used to translocate fusion proteins across a cell membrane. The fusion proteins can be conveniently fused to or derivatized with such sequences. Typically, the translocation sequence is provided as part of a fusion protein. Optionally, a linker can be used to link the fusion protein and the translocation sequence. Any suitable linker can be used, e.g., a peptide linker.

The fusion protein can also be introduced into an animal cell, preferably a mammalian cell, via liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell, i.e., the fusion protein.

The liposome fuses with the plasma membrane, thereby releasing the compound into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (e.g., the fusion protein or a nucleic acid encoding the same) at the target tissue or cell. For systemic or tissue specific delivery, this can be accomplished, for example, in a passive manner wherein the liposome bilayer degrades over time through the action of various agents in the body. Alternatively, active compound release involves using an agent to induce a permeability change in the liposome vesicle. Liposome membranes can be constructed so that they become destabilized when the environment becomes acidic near the liposome membrane (see, e.g., Proc. Natl. Acad. Sci. USA, 84:7851 (1987): Biochemistry, 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

Such liposomes typically comprise the fusion protein and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., 1980, Annu. Rev. Biophys. Bioeng., 9:467, U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication. No. WO 91/17424, Deamer & Bangham, 1976, Biochim. Biophys. Acta, 443:629-634; Fraley, et al., 1979, Proc. Natl. Acad. Sci. USA, 76:3348-52; Hope et al., 1985, Biochim. Biophys. Acta, 812:55-65: Mayer et al., 1986, Biochim. Biophys. Acta, 858:161-168; Williams et al., 1988, Proc. Natl. Acad. Sci. USA, 85:242-246; Liposomes (Ostro (ed.), 1983, Chapter 1): Hope et al., 1986, Chem. Phys. Lip., 40:89: Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments, it is desirable to target liposomes using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044).

Examples of targeting moieties include monoclonal antibodies specific to antigens associated with neoplasms, such as prostate cancer specific antigen and MAGE. Tumors can also be diagnosed by detecting gene products resulting from the activation or over-expression of oncogenes, such as ras or c-erbB2. In addition, many tumors express antigens normally expressed by fetal tissue, such as the alphafetoprotein (AFP) and carcinoembryonic antigen (CEA). Sites of viral infection can be diagnosed using various viral antigens such as hepatitis B core and surface antigens (HBVc, HBVs) hepatitis C antigens, Epstein-Barr virus antigens, human immunodeficiency type-1 virus (HIV1) and papilloma virus antigens. Inflammation can be detected using molecules specifically recognized by surface molecules which are expressed at sites of inflammation such as integrins (e.g., VCAM-1), selectin receptors (e.g., ELAM-1) and the like.

Standard methods for coupling targeting agents to liposomes can be used. These methods generally involve incorporation into liposomes lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see Renneisen et al., 1990, J. Biol. Chem., 265:16337-42 and Leonetti et al., 1990, Proc. Natl. Acad. Sci. USA, 87:2448-51).

### Dosages

For therapeutic applications, the dose of the fusion protein to be administered to a patient can be calculated in a similar way as has been described for zinc finger proteins, see for example U.S. Pat. Nos. 6,511,808, 6,492,117, 6,453,242, U.S. patent application 2002/0164575, and U.S. patent application 2002/0160940. In the context of the present disclosure, the dose should be sufficient to effect a beneficial therapeutic response in the patient over time. In addition, particular dosage regimens can be useful for determining phenotypic changes in an experimental setting, e.g., in functional genomics studies, and in cell or animal models. The dose will be determined by the efficacy, specificity, and Kp of the particular fusion protein employed, the nuclear volume of the target cell, and the condition of the patient, as well as the body weight or surface area of the patient to be treated. The size of the dose also will be determined by the existence, nature, and extent of any adverse side-effects that accompany the administration of a particular compound or vector in a particular patient.

### Pharmaceutical Compositions and Administration

Appropriate pharmaceutical compositions for administration of the fusion proteins of the present invention can be determined as described for zinc finger proteins, see for example U.S. Pat. Nos. 6,511,808, 6,492,117, 6,453,242, U.S. patent application 2002/0164575, and U.S. patent application 2002/0160940. Fusion proteins, and expression vectors encoding fusion proteins, can be administered directly to the patient for modulation of histone methylation patterns, e.g., and gene expression, and for therapeutic or prophylactic applications, for example, for treatment of diseases listed in associated with histone-mediated inhibition, including cancer (e.g., bladder, brain (e.g., glioma, or glioblastoma), breast, cervical, colon, colorectal, esophagus, head/neck, kidney, leukemia, liver, lung, lymphoma, myeloma, ovary, pancreas, prostate, rhabdomyosarcoma, and uterus cancer): schizophrenia: memory formation; and atherosclerosis. Thus the methods can include identifying a subject who has a disease associated with histone hypermethylation (e.g., optionally including obtaining a sample and detecting methylation of histones, e.g., of histones associated with a disease-associated gene, e.g., p14ARF, and selecting the subject if their sample includes hypermethylated histones), and administering a therapeutically effective amount of a fusion protein, or a nucleic acid encoding a fusion protein, as described herein, to the subject.

Administration of therapeutically effective amounts is by any of the routes normally used for introducing fusion proteins into ultimate contact with the tissue to be treated. The fusion proteins are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington: The Science and Practice of Pharmacy, 21st ed., 2005).

The fusion proteins, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

### EXAMPLES

The invention is further described in the following examples, which do not limit the scope of the invention described in the claims.

### Methods

The following materials and methods were used in the examples set forth below.

Construction of TALE fusions. The open reading frame for LSD1 was amplified from a cDNA library from K562 cells using primers (F:gttcaagatctttatctgggaagaaggcgg (SEQ ID NO:3), R:gaccttaattaaatgggcctcttcccttagaa (SEQ ID NO:4)). The PCR product was cloned into a TALE compatible expression vector27 using PacI and BamHI/BglII such that LSD1 was fused to the C-terminal end of the TALE. TALE repeat array monomers were designed and assembled using FLASH as described²⁴. These assembled DNA fragments were cloned into the expression vector using BsmBI sites and verified by restriction enzyme digestion and sequencing. The mCherry control vector was created by incorporating an mCherry open reading frame in place of the TALE array using Notl and PacI. Control TALE vectors lacking LSD1 were constructed using BamHI and PacI to remove LSD1, followed by blunt end ligation. The 3X Flag Tagged TALE vector was created by designing a gBlock (IDT) encoding a 29 amino acid Glycine:Serine linker followed by the 3X Flag sequence and cloning into the BamHI and PacI sites at the C-terminal end of the TALE repeat. Plasmids for construction of LSD1 and 3X Flag fusions will be available from Addgene.

Cell culture and transfection. The human erythroleukemia cell line, K562 (ATCC, CLL-243), was cultured in RPMI with 10% FBS, 1% Pen/Strep (Life Technologies). For transfection, 5 X 10⁶ cells per transfection were washed once with PBS. Cells were then transfected with 20 ug of TALE plasmid DNA or control mCherry plasmid by nucleofection with Lonza Kit V, as described by the manufacturer (Program T-016). Cells were immediately resuspended in K562 media at a cell density of 0.25 X 10⁶ cells/ml. Cells were harvested at 72 hours for ChIP or RNA extraction. For ZFPM2 gene expression analysis, the total amount of DNA per transfection was standardized by cotransfecting either 10 ug of a single TALE-LSD1 plasmid plus 10 ug of a scrambled TALE-LSD1 plasmid, or 10 ug each of two TALE-LSD1 plasmids. Transfection efficiency was determine by flow cytometry analysis of mCherry control transfected cells and ranged from 89-94% across multiple biological replicates.

Flag tagged ChIP. TALE-3X Flag transfected K562 cells were crosslinked with 0.5% formaldehyde for 5 minutes at room temperature. Nuclei were isolated and lysed as described²⁸. After sonication, solubilized chromatin was incubated with protein G Dynabeads (Invitrogen) and 0.5 ug anti-FLAG M2 antibody (Sigma) at 4° C. overnight. Samples were washed with TBS-T, low salt (150 mM NaCl, 2 mM Tris-HCl, 1% Triton-X), LiCl (250 mM LiCI, 1 mM Tris-HCl, 1% Triton-X), and high salt (750 mM NaCl, 2 mM Tris-HCl, 1% Triton-X) buffers at room temperature. Enriched chromatin was eluted (1% SDS, 5 mM DTT) at 65ºC for 20 minutes, purified and used directly for Illumina library prep. A control library was made from input DNA diluted to 50 picograms. Reads were aligned using Bowtie, and peak analysis was done using MACS with input controls, and masking genomic regions repetitive in Hg19 or K56229.

Native ChIP. Quantitative measurements of histone modification levels were performed in parallel using native ChIP. 0.01 U of MNase (ThermoScientific) was added to 1 ml lysis buffer (50 mM Tris-HCl, 150 mM NaCl, 1% Triton X-100, 0.1% sodium deoxycholate, ImM CaCl2) with EDTA free proteinase inhibitor. For each transfected sample, 260 ul of MNase: Lysis buffer was added and incubated for 15 minutes at 25ºC, and 20 minutes at 37ºC. MNase was inactivated by adding 20 mM EGTA. The lysed sample was split into 96 well plate format for ChIP with H3K4me2 (abcam ab32356) or H3K27ac (Active Motif 39133). Antibody binding, bead washing. DNA elution and sample clean-up were performed as described³⁰. ChIP DNA was analyzed by RT-PCR using FastStart Universal SYBR Green Master (Applied Biosystems), and enrichment ratios were calculated by comparison to equal amount of input DNA. Enrichment was normalized across ChIP samples to two standard off-target control enhancers (Table 2), and fold-ratios were calculated relative to mCherry plasmid transfected cells assayed in parallel. Each TALE ChIP experiment was performed in a minimum of 3 biological replicates. TALE-LSD1 reagents were scored based on the fold-changes of K4me2 and K27ac for two primers flanking the target sequence. A given reagent was scored as “effective” if it induced a 2-fold or greater reduction in modification signal for at least 2 of these 4 values, with a pvalue<0.05 using a one-tailed t-test. For ChIP-seq maps, 5 ng of ChIP DNA was used for library preparation as described³⁰.

Gene expression analysis. Genome-wide RNA expression analysis was performed using 3′DGE RNA-seq. Total RNA from 1 million TALE-LSD1 transfected or control (K562 alone or mCherry plasmid transfected) cells in biological replicate using RNeasy Mini kit (Qiagen). 2 ug of total RNA was fragmented and the 3′ ends of polyA mRNAs were isolated using Dynabeads (Invitrogen), and used to generate Illumina sequencing libraries, as described²⁵. To precisely quantify the gene expression, a 3′ DGE analysis pipeline was used. The pipeline estimates gene expression based on the maximum number of reads in any 500 basepair window within 10 kb of the annotated 3′ gene end. This approach compensates for the fact that annotated ends for some genes are imprecise and may be cell type dependent and yields accurate quantifications. We then normalized the gene expression levels, scaling samples by the median gene inter-sample variation, as described in26. This approach controls for differences in sequencing depth between libraries and in the overall transcript abundance distribution.

The 22 RNA-seq datasets were then normalized based on their negative binomial distributions. Libraries with extreme normalization coefficients below 0.7 or above 1.5 were excluded. To identify candidate regulated genes, the three closest upstream and three closest downstream genes were examined. A gene was specifically scored as regulated if (i) it was detected in control K562 cells with a normalized RNA-seq value >10, i.e. the top 50th percentile of expression: (ii) its mean expression value was at least 1.5-fold lower in the corresponding on-target TALE-LSD1 libraries compared to all other libraries, p<0.05 calculated using DESeq26 and (iii) its normalized 3′DGE values in the on-target TALE-LSD1 libraries were the two lowest over all 22 datasets. To simulate the 1000 random binding sites, we sample genomic positions uniformly at random and use rejection sampling to ensure that the random set has a similar distribution relative to genomic annotations (intergenic, promoter, gene body, UTR) to the actual TALE binding sites. We then used significance testing criteria identical to that applied to the actual TALE experiments.

For RT-PCR based expression analysis, total RNA was extracted and reverse transcribed into cDNA using Superscript III First-Strand Synthesis system for RT-PCR (Invitrogen). Quantitative PCR was performed with FastStart Universal SYBR Green Master (Invitrogen) with primer sequences listed in Table 2 on an ABI 7500 machine. Gene expression values are presented as log 2 Ct ratios relative to 2 housekeeping control genes (TBP and SDHA). and represents an average of four independent biological replicates each assayed in two technical replicates.

TABLE 2
Primer Sequences Used

| | ChIP | | | | |
|---|---|---|---|---|---|
| | qPCR | | SEQ | | SEQ |
| TALE | Primer | | ID | | ID |
| ID # | Set | F | NO: | R | NO: |
| 1 | 1.1 | GGAATCGTGAATACCCCTGA | 47 | AACATGCAGGTCTGCTTTCC | 48 |
| 1 | 1.2 | GGAATTGGCCTGCAGAATTA | 49 | GTACACCATTGGCTGGCTCT | 50 |
| 2 | 2.1 | TACTGACCCATGAGCACAGC | 51 | CCCCACTGCCATCCTACTTA | 52 |
| 2 | 2.2 | GAGTGTTGGCAGAATGAGCA | 53 | TGTGCGTATGCATTTTGTTCT | 54 |
| 3 | 3.1 | AGCACACAATTTTGCTCATCA | 55 | ACGTGCACATGGAACAAGAC | 56 |
| 3 | 3.2 | CTGCCAAGTTTCTGGTTGGT | 57 | GAGACAAAATAGCGGGGACA | 58 |
| 4 | 4.1 | AAGAGGACATTCTGGGCTGA | 59 | CCTGCCTCCTAAGCTTCCTT | 60 |
| 4 | 4.2 | GACCTGACTCGAACCCACTC | 61 | GCCTCTGCTAAGGCACAAAC | 62 |
| 5 | 5.1 | TGCCTAGGAAGGCACTTGTC | 63 | GGCTGGAGATCAGCTTTTTG | 64 |
| 5 | 5.1 | TGTCCTGGAACGGTTTCACT | 65 | TTTCTCCTTTGGGCATCTTG | 66 |
| 6 | 6.1 | AAGAGGACATTCTGGGCTGA | 67 | CCTGCCTCCTAAGCTTCCTT | 68 |
| 6 | 6.2 | GACCTGACTCGAACCCACTC | 69 | GCCTCTGCTAAGGCACAAAC | 70 |
| 7 | 7.1 | CCCTTGACCAGGTAGGTTCA | 71 | AAGGAGGGCTCCAGTTTCAT | 72 |
| 7 | 7.2 | TGGTGGAATGAGTAGCAGAGC | 73 | GGGGATTTTCACACTTGGTG | 74 |
| 8 | 8.1 | TGTCTGCACAAATTGCTGTG | 75 | CTTGGGAGGGGTTCAGAGAC | 76 |
| 8 | 8.2 | ACTCAAAGGTGGGTGTGAGG | 77 | TCCGATAATCTGGTCCAAGG | 78 |
| 9 | 9.1 | CCCAGGAAACTTGATGAGAGA | 79 | TGTGGAAGGAGTGAGTGAACA | 80 |
| 9 | 9.2 | GGGTTTTCATGAAGCTTTGAA | 81 | TTTCGTATTGCATCCCATCA | 82 |
| 10 | 10.1 | GCTGAGCTTTTCAGGTAGGC | 83 | GCTCCCAAAAAGATGCAAGT | 84 |
| 10 | 10.2 | GGGCCCTCCTTATACTTGGA | 85 | TGGACTGGGAGGAACATAGC | 86 |
| 11 | 11.1 | TGCTACGTGCAGCGTATTCT | 87 | TGCAACGCTATTTCTCAGGA | 88 |
| 11 | 11.2 | AGCATTTTCAGCCTCAGTGG | 89 | CCTTGTAGCACCTCTGTCCA | 90 |
| 12 | 12.1 | CAGACTTCTGGAACGCAGTG | 91 | TGTGACAGGCCAAGTCTCAG | 92 |
| 12 | 12.2 | CTGACGGTTTATGAGCAGCA | 93 | GTTTCCCACAGTTCCCTGAA | 94 |
| 13 | 13.1 | TGAAGTCCACATGTTTAGCTCCT | 95 | TGGAAGGAATGTGATTCCACT | 96 |
| 13 | 13.2 | TTCAACAGCAACCAGGAATG | 97 | AAGCTCAAAAAGAAAAACTTCAACA | 98 |
| 14 | 14.1 | CCATTTTCCGTACATGGTGA | 99 | CTGGCTGTAGGGCTCTGTTC | 100 |
| 14 | 14.2 | GACGGGGAAGGAAGAAAGAA | 101 | TCCCAGCTCTCGCAGCTT | 102 |
| 15 | 15.1 | TACACAACAGCACCCACACA | 103 | CCCCATTTCAGTTCTTTCTCA | 104 |
| 15 | 15.2 | TCTTCTGGGTTTGTTGGCTA | 105 | GGCACCATGTGAACTCTCCT | 106 |
| 16 | 16.1 | TCCAACTCAATGCCTTTTCTG | 107 | CACAGGCAAGATTCCCATTT | 108 |
| 16 | 16.2 | AATGGCTCTGGAGAAAAGCA | 109 | GCATGCCAGTCTGAAGATGA | 110 |
| 17 | 17.1 | TGTGAACCTCGAGAAGTGTGA | 111 | TTGTTGAGGTGTGCATGAGG | 112 |
| 17 | 17.2 | GTCATGTCCAGCAGGATGC | 113 | ATGCAGCTGACCCATTGTTT | 114 |
| 18 | 18.1 | ACGATGGAGGACATTGGAAG | 115 | TGAAGGCTTTTCAGGAGCTT | 116 |
| 18 | 18.2 | CTGCAAACAAGGTCTTTGGAC | 117 | AGGCAGCTACCTGGTTAAGG | 118 |
| 19 | 19.1 | GTGACCTTGGAGACGTTGCT | 119 | AGCCTCTTGAACCAGAGCAG | 120 |
| 19 | 19.2 | AAGAGAAGGAGAACCAAGCCTTA | 121 | CACACCAGCAAAGAGCAAAA | 122 |
| 20 | 20.1 | GATTCCGGGTCACTGTGAGT | 123 | TTTTACGGCGAGATGGTTTC | 124 |
| 21 | 21.1 | GGAAGAAAGGAAGGTAGGAAGG | 125 | AGGGCACTCTCCTCTCCTCT | 126 |
| 21 | 21.2 | GCTGAGACCACCCACTCTTC | 127 | CCCAGAAGGAATTACCCACA | 128 |
| 22 | 22.1 | TCACACATCACTTGCGTTCA | 129 | TGGCTTGATAACCCAACCAT | 130 |
| 22 | 22.2 | AGGGAGCACTCTAGGGATGG | 131 | CAGGGGAAACAGGAAGTGAG | 132 |
| 23 | 23.1 | CCACTAAACCGCAACCAAAG | 133 | GGAAACTCCCAGCTTTCAAAC | 134 |
| 23 | 23.2 | CGTTTCTCCCTGGGTTCTTT | 135 | ATTTTTCTGCCTCCCAAACC | 136 |
| 24 | 24.1 | CTGCCCCCAAAGAAAGGTAT | 137 | TTGGCATACTTCATGCTCACA | 138 |
| 24 | 24.2 | TTGACATTAGGTCCAGGTTTGA | 139 | TATTTTAGGGCAGGCACACC | 140 |
| 25 | 25.1 | TCATTTTGGTAGCCTTTCTGC | 141 | CACTCAAGTCCCAGGTTGGT | 142 |
| 25 | 25.2 | GATGATTTGGCTTTTGCGATA | 143 | CTTGTGGGAGCTCGACATTA | 144 |
| 26 | 26.1 | GACGTGTTGGTGCATACCTG | 145 | ATGAGGCTCCTCCCTCATTT | 146 |
| 26 | 26.2 | TCAAGAGTACGGCAATCACG | 147 | GGGAAACCGAAGGATTGATT | 148 |
| 27 | 27.1 | GACCACCGGTCTTCTCATGT | 149 | GCAGCTGATGAAGAGCAGAA | 150 |
| 27 | 27.2 | TAGGGTGTGGATGTGGAACA | 151 | TGGGAAATTGCTGTGTTGAG | 152 |
| 28 | 28.1 | TCCTGTAAAGTCCTCAGATCAACA | 153 | GCCAGCTTCTAAGGATGCAC | 154 |
| 28 | 28.2 | TTGGTCTTTGGCCTTCTAGG | 155 | AATGGGGAAGTGACAAGGAA | 156 |
| 29 | 29.1 | CAGCCTTTCTAGGAATCACAAA | 157 | GGATGATGAGGAACTGGCTTT | 158 |
| 30 | 30.1 | GTGAACCACCAAGCACAGC | 159 | AGCAGGGGTGGAGAGAAAAT | 160 |
| 30 | 30.2 | GGCTACAGCGTCTTCCTGTG | 161 | CACACACCACACCCACAACT | 162 |
| 31 | 31.1 | TAAGGCCGGTCTATCACAGC | 163 | GCAGTCTCAGCACCTCAACC | 164 |
| 31 | 31.2 | ACTGCCTGCCTGGAGTCTAC | 165 | TCGCTCACTGAGGAATGATG | 166 |
| 32 | 32.1 | TACACCGCGAAGGGATAGTC | 167 | TGGGGGTCAGAGAGAGAATG | 168 |
| 33 | 33.1 | GGGCCCCAGACTTTAATTTG | 169 | GCCTCTGGAGTGCAGTACCT | 170 |
| 33 | 33.2 | CCCAGATATTTCCTGCTCCA | 171 | CCCCCAAATTCCATTATTCC | 172 |
| 34 | 34.1 | GAGGGAGCGAGCCATAGTG | 173 | ACAATGGGGCTGCCTGAG | 174 |
| 34 | 34.2 | GGAGGAGGGTGGTCTCTCAT | 175 | TCGAAAGCTACACGGCTCTT | 176 |
| 35 | 35.1 | TGGGTGAGGAAGGAGAAAGA | 177 | AAACCCCTATGGGCAACTCT | 178 |
| 36 | 36.1 | CTGGCCCTCTTCTCCTTTCT | 179 | CAATCATTTGCCAACACAGG | 180 |
| 36 | 36.2 | GTCTGAGGAAAGGCACCTGA | 181 | TCGCACCTGTGTGAGAGGTA | 182 |
| 37 | 37.1 | AGCGACAAAAGGTCAACAGA | 183 | GGTGTTGCGGAAAACACTTT | 184 |
| 37 | 37.2 | CCTAAGAATCAGAAACGCAATG | 185 | CAGTCTGGGCAACAGAACAA | 186 |
| 38 | 38.1 | AACGAAACACAACCTGCACA | 187 | CTGTAACCCTACCCCCAACC | 188 |
| 38 | 38.2 | CAGAACAAAATGGAGTCTTAGCC | 189 | TCAGAAGGTGTGGGGAAAAG | 190 |
| 39 | 39.1 | ATGGCTTTCATGAAGCTGGA | 191 | CGTCTGTGCGAAGAGAAGC | 192 |
| 39 | 39.2 | AAAGCATTTTTGCCATCCAG | 193 | TTCCCGGTTAGATGAGTTGG | 194 |
| 40 | 40.1 | GCCCTCCCTTGATAAGAACC | 195 | TGGGAACCTCTCCATCTCAC | 196 |
| 40 | 40.1 | CCAAAGTCACATGGATGACAG | 197 | GGCTAAATGAGGCAGATGCT | 198 |

| | cDNA | | | | |
|---|---|---|---|---|---|
| | qPCR | | | | |
| TALE | Primer | | | | |
| ID # | Set | F | | R | |
| 14 | GPKOW | CTGAGGGAAGACATGCTGGA | 199 | AGTGAAGCTCCACCACCTGA | 200 |
| | MAGIX | CCCAGCTCCACCTGGTTATT | 201 | CTAGGGAAGTGCTGCTGCTG | 202 |
| | PLP2 | ATGTGTGACCTGCACACCAA | 203 | CTTTACCCCTGCGACGATTT | 204 |
| | PRICKLE3 | GGCACCAGCACAGAGTTAGC | 205 | GACGACCGAAGGCACTATCA | 206 |
| 25 | LRP12 | GAAGCTCCTCCCTCGTATGG | 207 | TCCAAGCTGAGATCGTACCG | 208 |
| | ZFPM2.1 | ATCAGATTTCCAGCCTGTGC | 209 | TGATCACGGAATCAGCAGTG | 210 |
| | ANGPT1 | CTGGGACAGCAGGAAAACAG | 211 | TAGATTGGAGGGGCCACAAG | 212 |
| | ZFPM2.2 | GGCCTGAAAATCTGAGCTGC | 213 | CAGTCGTCTGTCTCAACTCCA | 214 |
| | ZFPM2.3 | GTACAGCAAAGGGGGTCAGC | 215 | GACTGGCAGCTTGTAGCCTT | 216 |
| | ZFPM2.4 | GTTTTATCTTTTGAAAGGCACAGTC | 217 | TTGTGATCACCAGGTGCAGT | 218 |
| | ZFPM2.5 | TCAATTCAGCTGCTTCCTCA | 219 | CTGGAAATCTGATGGGCACT | 220 |
| | SDHA | TCTGCACTCTGGGGAAGAAG | 221 | CAAGAATGAAGCAAGGGACA | 222 |
| | TBP | TTCCCCATGAACCACAGTTT | 223 | TGCAATACTGGAGAGGTGGA | 224 |

### Example 1

Initial experiments focused on a candidate enhancer in the stem cell leukemia (SCL) locus that is enriched for H3K4me2 and H3K27ac in K562 erythroleukemia cells4,6,9,12,13,19 SCL encodes a developmental transcription factor with critical functions in hematopoiesis that is expressed in K562 cells. A TALE array was designed to bind an 18 base sequence in a segment of this enhancer predicted to be nucleosome-free based on DNase hypersensitivity (FIG. 1A). Since the binding specificity of monomeric TALEs has yet to be thoroughly characterized, an expression construct encoding this TALE array fused to a 3X FLAG epitope was first created. This construct was transfected into K562 cells, expression confirmed by Western blot, and genome-wide binding mapped by chromatin immunoprecipitation and sequencing (ChIP-seq). The top ranked binding site corresponded precisely to the target sequence within the SCL locus (FIG. 1B, Table 3). No other ChIP-seq peaks were reproducibly detected in the two biological replicates.

TABLE 3
TALE-3X Flag ChIP-seq Peaks

| Chr location | Rep1 tags | Rep2 tags | pvalue |
|---|---|---|---|
| chr1: 47, 646, 591-47, 647, 590 | 25 | 20 | 0.01 |
| chr1: 17, 221, 975-17, 222, 974 | 3 | 8 | 0.14 |
| chr5: 78, 850, 956-78, 851, 955 | 5 | 1 | 0.21 |
| chr17: 51, 183, 234-51, 184, 233 | 2 | 4 | 0.15 |

Peak calls using MACS in two biologically independent replicates along with reads falling within a 1 kb window around the peak. Grey shading indicates the target locus. P-values calculated by comparison of both biological replicates to the input control library.

The genome was scanned for sequence motifs with one or two mismatches from the TALE recognition motif, but no significant ChIP-seq enrichments were detected at these sites either (Table 4).

TABLE 4

| | TALE-3X Flag | |
|---|---|---|
| Target Sequence | ChIP tags per 1 kb bin | Input tags per 1 kb bin |
| 18/18 Target (n = 1) | 17.5 | 1 |
| 17/18 Targets (n = 2) | 0.5 | 0.5 |
| 16/18 Targets (n = 52) | 0.40 | 0.58 |

The sequence read count at 54 genomic loci with 1 or 2 mismatches compared to the perfect match target locus for the TALE-3X Flag.

These data support the specificity of TALE binding and are consistent with prior demonstrations of TALE activator domain fusions that selectively induce target genes 14.18.20

### Example 2

To modulate chromatin state at the SCL enhancer, the corresponding TALE was combined with the LSD1 demethylase. K562 cells were transfected with a construct encoding this TALE-LSD1 fusion or a control mCherry vector, the cells cultured for three days and histone modification levels measured by ChIPqPCR.

The fusion reduced H3K4me2 signals at the target locus by ˜3-fold relative to control, but had no effect at several non-target control enhancers (FIGS. 1C and 1E). In addition to its enzymatic activity, LSD1 physically interacts with other chromatin modifying enzymes, including histone deacetylases21. Therefore changes in H3K27ac, another characteristic enhancer mark, were also assayed. The fusion reduced H3K27ac levels by >4-fold, suggesting that LSD1 recruitment leads to generalized chromatin inactivation at the target enhancer.

### Example 3

To eliminate the possibility that the chromatin changes reflect displacement of other transcription factors by the TALE, a construct encoding the TALE without LSD1 was tested. A TALE-LSD1 fusion with a scrambled target sequence not present in the human genome was also examined to control for non-specific effects of LSD1 overexpression. Neither construct altered H3K4me2 or H3K27ac levels at the SCL locus (FIGS. 1C and 1F).

Lastly, to evaluate the specificity of the fusion comprehensively, ChIP-seq was used to map H3K4me2 and H3K27ac genome-wide in TALE-LSD1 and control transfected K562 cells. These data confirmed loss of H3K4me2 and H3K27ac across a 2 kb region surrounding the target sequence within the SCL locus (FIG. 1D).

These results indicate that directed LSD1 recruitment results in locus-specific reduction of H3K4me2 and H3K27ac. The generalized effect on chromatin state may be a direct consequence of H3K4 demethylation or, alternatively, may depend on partner proteins that associate with LSD 115.16.22.23. Regardless, prior studies indicate that sequence elements enriched for H3K4me2 and H3K27ac exhibit enhancer activity in corresponding cell types, while elements lacking these marks are rarely active^{4.6.12}. Hence, these results suggest that this TALE-LSD1 fusion efficiently and selectively inactivates its target enhancer.

### Example 4

The study was expanded to investigate a larger set of candidate enhancers with active chromatin in K562 cells. These include nine elements in developmental loci, sixteen additional highly cell type-specific elements, and fifteen intergenic elements. TALE repeat arrays were designed and produced for sequences in these 40 enhancers using the Fast Ligation-based Automatable Solid-phase High-throughput (FLASH) assembly method24 (Table 1). LSD1 fusion constructs were then cloned for each TALE and transfected individually into K562 cells, alongside mCherry control plasmid transfected separately into cells. At three days post transfection, H3K4me2 and H3K27ac were measured by ChIPqPCR using two primer sets per target enhancer.

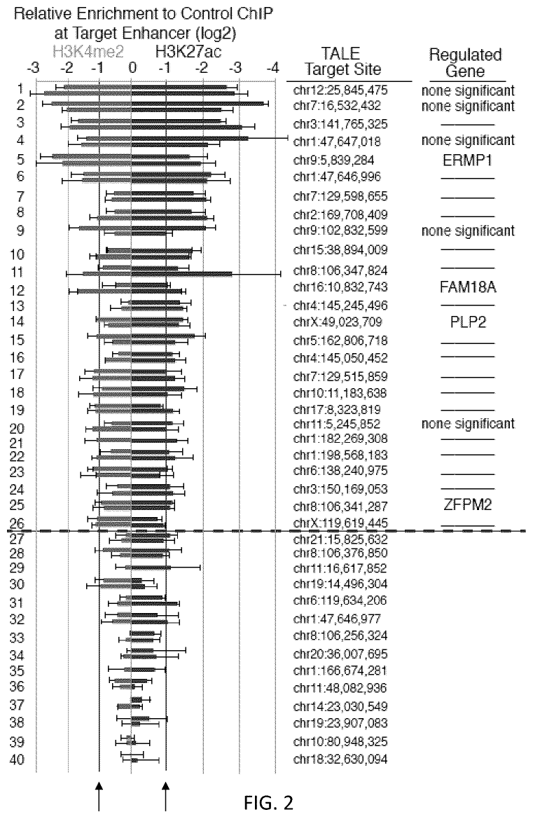

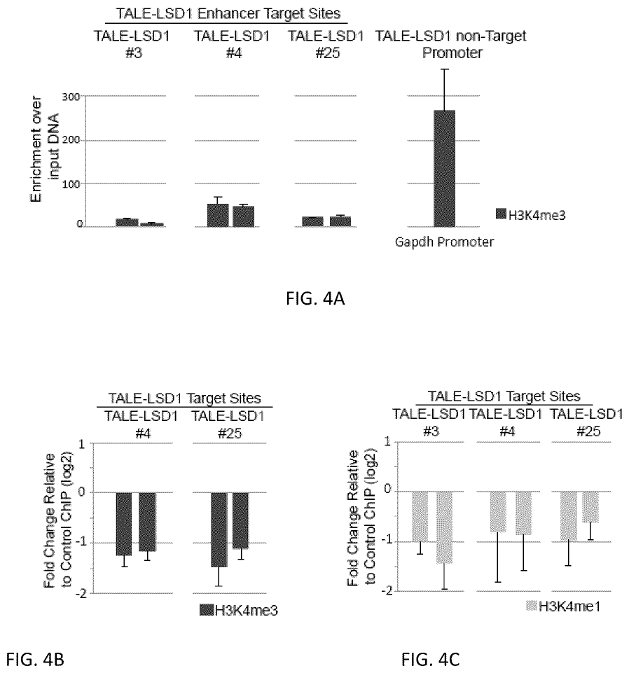

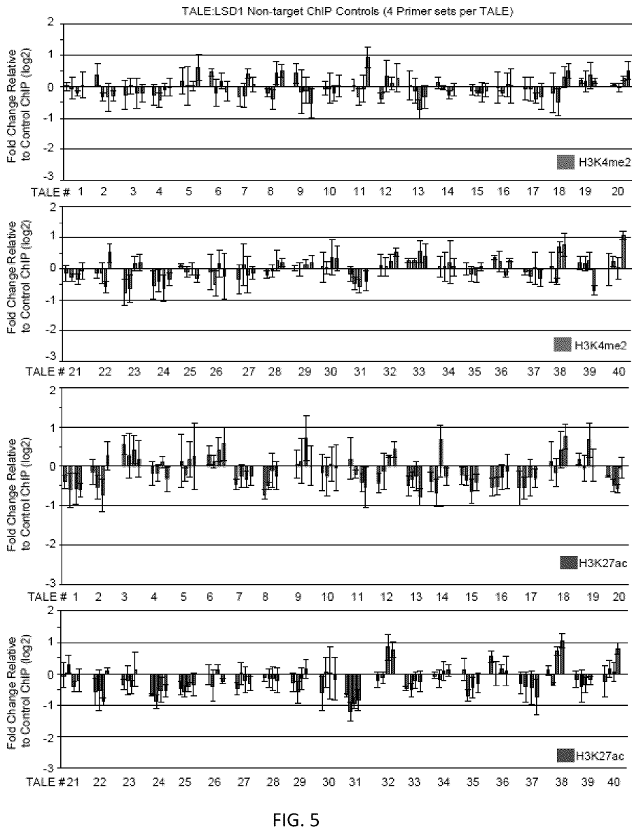

26 of the 40 TALE-LSD1 constructs (65%) significantly reduced levels of these modifications at their target loci, relative to control transfected cells (FIG. 2). An additional 8 constructs caused more modest reductions at their targets, suggesting that the strategy can be effective at most enhancers (FIG. 2). ChIP-qPCR measurements of H3K4me1 and H3K4me3 confirm that the reagents also reduce these alternative H3K4 methylation states (FIGS. 4A-C). The induced changes were specific to the target loci, as analogous measurements at non-target enhancers did not reveal substantial changes (FIG. 5). Furthermore, genome-wide ChIP-seq analysis of two TALE-LSD1 fusions that were positive by ChIPqPCR confirmed the robustness and specificity with which they reduce chromatin signals at target loci (FIG. 6). These results suggest that TALE-LSD1 fusions can provide an effective means for inactivating chromatin at any target enhancer.

TABLE 1
TALE Array Target Sequences

| TALE | | TALE | |
|---|---|---|---|
| ID# | chr # | Target Sequence | SEQ ID NO: |
| 1 | chr12:25,845,475 | TTCAGTTGTGGTATCTG | 6 |
| 2 | chr7:16,532,432 | TACCATGTCTTTCTAAG | 7 |
| 3 | chr3:141,765,325 | TTTACAGAGCTGTGGTCACT | 8 |
| 4 | chr1:47,647,018 | TCCGTGGCTGCCAGTCTG | 9 |
| 5 | chr9:5,839,284 | TGCATATACTTTTTAATG | 10 |
| 6 | chr1:47,646,996 | TCCAGGAGCGCGCCTGAG | 11 |
| 7 | chr7:129,598,655 | TGCCTGTGAGGAACAGCTGT | 12 |
| 8 | chr2:169,708,409 | TGCAGACATCTCCAGGCTCT | 13 |
| 9 | chr9:102,832,599 | TAATTTGTACATGGTTACAT | 14 |
| 10 | chr15:38,894,009 | TGTTAGTTACCATATTGTGG | 15 |
| 11 | chr8:106,347,824 | TCCAGTCCCTGGCTCCCATG | 16 |
| 12 | chr16:10,832,743 | TGGCTAATTTTTGGTATTTT | 17 |
| 13 | chr4:145,245,496 | TGGCTTTCCTTCCCTTTG | 18 |
| 14 | chrX:49,023,709 | TAGCCGCGAGGAAGGCG | 19 |
| 15 | chr5:162,806,718 | TAAAGACCTGTTACCCAATT | 20 |
| 16 | chr4:145,050,452 | TCGTTTTTCTTTTTTGGAAG | 21 |
| 17 | chr7:129,515,859 | TTCTAAATTGAGGTGCTG | 22 |
| 18 | chr10:11,183,638 | TCAATCATTGCATGTTTATT | 23 |
| 19 | chr17:8,323,819 | TTGCATCTGGGACAGATG | 24 |
| 20 | chr11:5,245,852 | TTGATGGTAACACTATG | 25 |
| 21 | chr1:182,269,308 | TTATCTCCCTCACCCAG | 26 |
| 22 | chr1:198,568,183 | TGGTTAGAAACACAGCTGCC | 27 |
| 23 | chr6:138,240,975 | TTCATGGTTCAATAAAGACT | 28 |
| 24 | chr3:150,169,053 | TACATAAAATTTTTAAGG | 29 |
| 25 | chr8:106,341,287 | TTAAGCTTCTGAAGTCAG | 30 |
| 26 | chrX:119,619,445 | TGATCTTCATTTTTAAAG | 31 |
| 27 | chr21:15,825,632 | TGGTATGAGTTGAAAATG | 32 |
| 28 | chr8:106,376,850 | TAAGTCTACATATAGTATCC | 33 |
| 29 | chr11:16,617,852 | TAAAATGCACTCACAATG | 34 |
| 30 | chr19:14,496,304 | TCTCTGAATCCCCTGGTGAC | 35 |
| 31 | chr6:119,634,206 | TTAAACAGATAAGGGAG | 36 |
| 32 | chr1:47,646,977 | TGGTGCGTTATCAGCCTT | 37 |
| 33 | chr8:106,256,324 | TCAATACCCCACAAAGAAGC | 38 |
| 34 | chr20:36,007,695 | TCTCTACCTTGGAGGCTG | 39 |
| 35 | chr1:166,674,281 | TAGAAAATACAACCTCAG | 40 |
| 36 | chr11:48,082,936 | TCCTGGAAAAGCCCTCTATG | 41 |
| 37 | chr14:23,030,549 | TAAGTTTGCAAACAAGCTCC | 42 |
| 38 | chr19:23,907,083 | TGGCTTTCCTAGGCAGAAGT | 43 |
| 39 | chr10:80,948,325 | TCACGCCTTTGTGGCCAGAG | 44 |
| 40 | chr18:32,630,094 | TCACTGTGTACCTTTTTATG | 45 |
| non- | N/A | TGCAGTGCTTCAGCCGCT | 46 |
| Target | | | |

### Example 5

Next, whether reduced chromatin activity at specific enhancers affects the transcriptional output of nearby genes was considered. These experiments initially focused on 9 TALE-LSD1 fusions that robustly alter chromatin state (FIG. 2), and systematically screened for regulated genes using a modified RNA-seq procedure termed 3′ Digital Gene Expression (3′DGE). By only sequencing the 3′ ends of mRNAs, this procedure enables quantitative analysis of transcript levels at modest sequencing depths25 as described above. A gene was scored as regulated if (i) it was detected in control K562 cells with a normalized RNA-seq value >10, i.e. the top 50th percentile of expression; (ii) its mean expression value was at least 1.5-fold lower in the corresponding on-target TALE-LSD1 libraries compared to all other libraries, p<0.05 calculated using DESeq26 and (iii) its normalized 3′DGE values in the on-target TALE-LSD1 libraries were the two lowest over all 22 datasets.

The 9 TALEs were transfected individually into K562 cells, alongside with control mCherry plasmids and measured mRNA levels in biological replicate. Each 3′DGE dataset was normalized based on a negative binomial distribution and excluded any libraries that did not satisfy quality controls as described above and in26. Whether any of the TALE-LSD1 reagents significantly altered the expression of genes in the vicinity of its target enhancer was then examined.

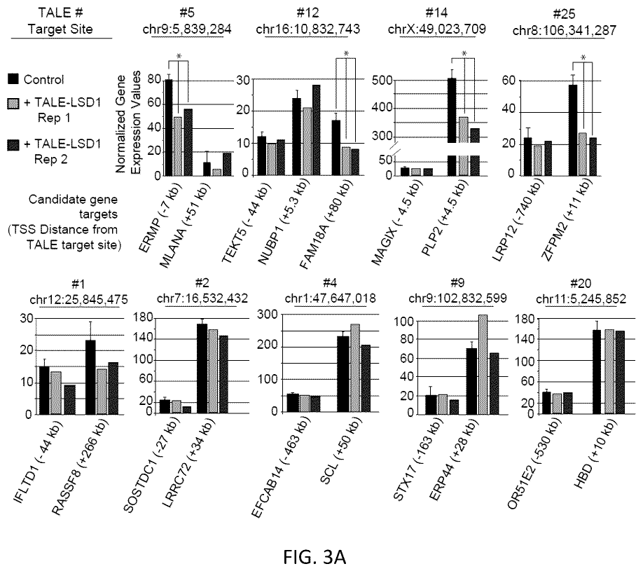

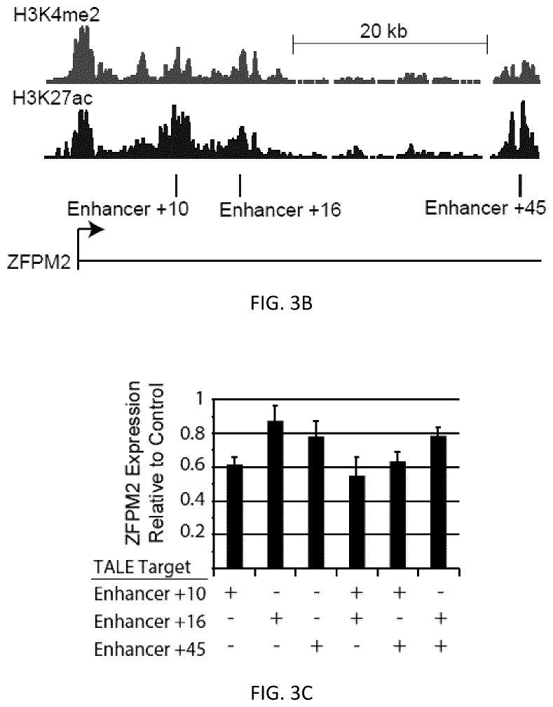

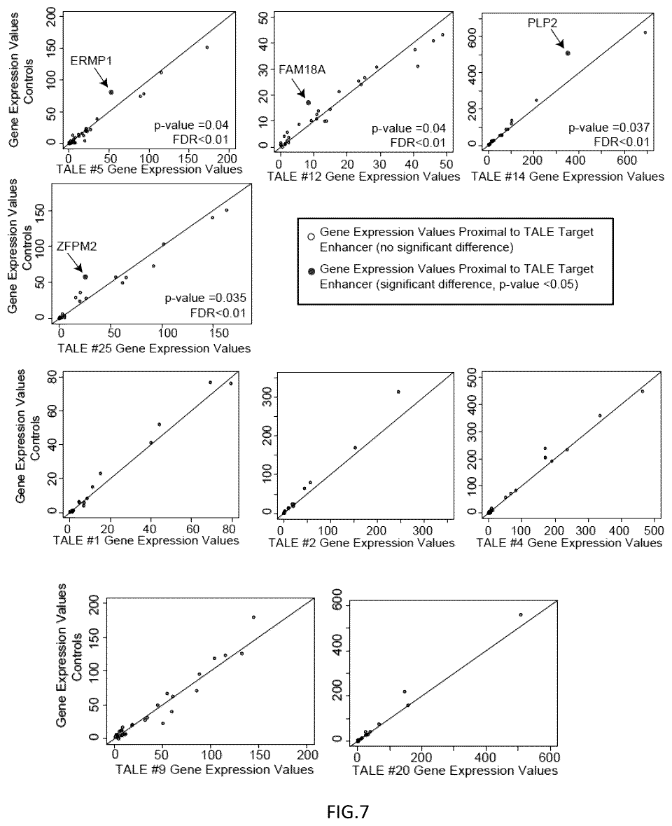

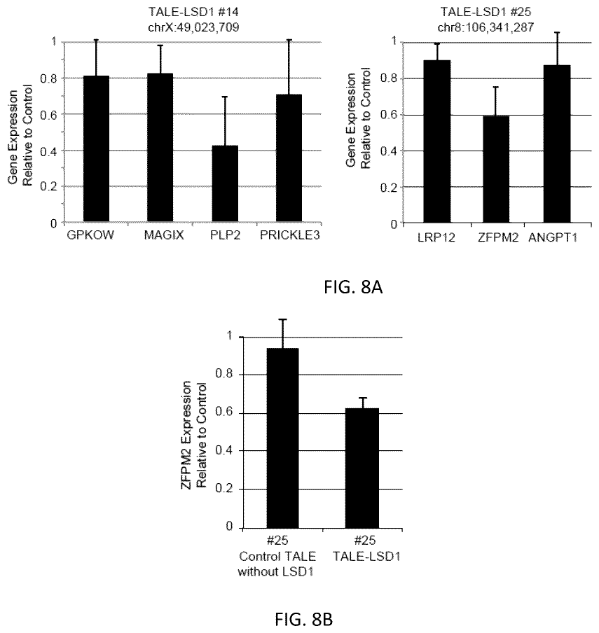

Four of the nine tested fusions (44%) caused a nearby gene to be down-regulated by at least 1.5-fold, with both biological replicates representing the two outlying values across all 22 RNA-seq datasets (see Methods, FIG. 3A, FIG. 7). The significance of these transcriptional changes is supported by a simulated analysis of a random sampling of 1000 genomic locations that did not yield any false-positives in which an adjacent gene scored as regulated (FDR<0).1%). The expression changes were also confirmed by quantitative RT-PCR (FIG. 8A). Two of the enhancers that significantly regulated genes are intergenic, while a third coincides with the 3′ end of a gene, but affects the activity of the next downstream gene. The fourth scoring enhancer resides in the first intron of ZFPM2. A TALE lacking the demethylase did not affect ZFPM2 expression, confirming that ZFPM2 down-regulation requires LSD1 recruitment (FIG. 8B). It was not possible to distinguish whether the other five putative enhancers have weak transcriptional effects below the detection threshold or, alternatively, do not regulate any genes in K562 cells. Regardless, these results indicate that TALE-LSD1 fusions can alter enhancer activity in a targeted, loss-of-function manner, and thereby enable identification and modulation of their target genes.

### Example 6

The high prevalence of putative enhancers in the genome suggests that many act redundantly or function only in specific contexts, which could explain our inability to assign target genes to roughly half of the tested elements. To address the former, three putative enhancers were examined within the developmental locus encoding ZFPM2 (FIG. 3B). In addition to the TALE-LSD1 fusion targeted to the intronic enhancer described above (FIG. 3A, 3B: enhancer+10), TALE-LSD1 fusions were designed and validated that reduced modification levels at two additional intronic ZFPM2 enhancers (enhancers+16, +45) (FIG. 2, 3B). First, each TALE-LSD1 fusion was transfected individually and their effects on ZFPM2 expression tested by qPCR. While the fusion targeting the original+10 enhancer reduced ZFPM2 expression by ˜2-fold, the fusions targeting the +16 and +45 enhancers showed only modest reductions of ˜13% and ˜22%, respectively, which did not reach statistical significance (FIG. 3C). To determine if these enhancers act additively or synergistically, the fusions were transfected in pairwise combinations. Although targeting pairs of enhancers tended to reduce gene expression more than hitting a single enhancer, the cumulative effects were substantially less than the sum of the two individual effects. This suggests that the multiple enhancers in this locus function redundantly to maintain ZFPM2 expression in K562 cells. These results indicate the potential of programmable TALE-LSD1 fusions to shed light on complex regulatory interactions among multiple enhancers and genes in a locus.

### REFERENCES

1. Bulger, M. & Groudine, M. Functional and mechanistic diversity of distal transcription enhancers. Cell 144, 327-339 (2011).
2. Visel, A., Rubin, E. M. & Pennacchio, L. A. Genomic views of distant-acting enhancers. Nature 461, 199-205 (2009).
3. Noonan, J. P. & McCallion, A. S. Genomics of long-range regulatory elements. Annu Rev Genomics Hum Genet 11, 1-23 (2010).
4. Heintzman, N. D. et al. Histone modifications at human enhancers reflect global cell-type-specific gene expression. Nature 459, 108-112 (2009).
5. Boyle, A. P. et al. High-resolution mapping and characterization of open chromatin across the genome. Cell 132, 311-322 (2008).
6. Ernst, J. et al. Mapping and analysis of chromatin state dynamics in nine human cell types. Nature 473, 43-49 (2011).
7. Consortium, T. E. P. et al. An integrated encyclopedia of DNA elements in the human genome. Nature 488, 57-74 (2012).
8. Maurano, M. T. et al. Systematic localization of common disease-associated variation in regulatory DNA. Science 337, 1190-1195 (2012).
9. Calo, E. & Wysocka, J. Modification of Enhancer Chromatin: What, How, and Why? MOLCEL 49, 825-837 (2013).
10. Stadler, M. B. et al. DNA-binding factors shape the mouse methylome at distal regulatory regions. Nature 480, 490-495 (2011).
11. Ng. J.- H. et al. In vivo epigenomic profiling of germ cells reveals germ cell molecular signatures. Dev Cell 24, 324-333 (2013).
12. Creyghton, M. P. et al. Histone H3K27ac separates active from poised enhancers and predicts developmental state. Proceedings of the National Academy of Sciences 107, 21931-21936 (2010).
13. Rada-Iglesias, A. et al. A unique chromatin signature uncovers early developmental enhancers in humans. Nature 470, 279-283 (2011).
14. Shi, Y. et al. Histone Demethylation Mediated by the Nuclear Amine Oxidase Homolog LSD1. Cell 119, 941-953 (2004).
15. Boch, J. et al. Breaking the Code of DNA Binding Specificity of TAL-Type III Effectors. Science 326, 1509-1512 (2009).
16. Moscou, M. J. & Bogdanove, A. J. A simple cipher governs DNA recognition by TAL effectors. Science 326, 1501 (2009).
17. Mussolino, C. & Cathomen, T. TALE nucleases: tailored genome engineering made easy. Curr Opin Biotechnol 23, 644-650 (2012).
18. Joung, J. K. & Sander, J. D. TALENs: a widely applicable technology for targeted genome editing. Nat. Rev. Mol. Cell Biol. 14, 49-55 (2013).
19. Dhami, P. et al. Genomic Approaches Uncover Increasing Complexities in the Regulatory Landscape at the Human SCL (TAL1) Locus. PLOS ONE 5, e9059 (2010).
20. Zhang, F. et al. Efficient construction of sequence-specific TAL effectors for modulating mammalian transcription. Nat Biotechnol 29, 149-153 (2011).
21 Lee, M. G., Wynder, C., Cooch, N. & Shiekhattar, R. An essential role for CoREST in nucleosomal histone 3 lysine 4 demethylation. Nature (2005). doi: 10.1038/nature04021
22. Whyte, W. A. et al. Enhancer decommissioning by LSD1 during embryonic stem cell differentiation. Nature 1-5 (2012). doi: 10.1038/nature10805
23. Reyon, D. et al. FLASH assembly of TALENs for high-throughput genome editing. Nat Biotechnol 30, 460-465 (2012).
24. Yoon, O. K. & Brem, R. B. Noncanonical transcript forms in yeast and their regulation during environmental stress. RNA 16, 1256-1267 (2010).
25. Anders, S. & Huber, W. Differential expression analysis for sequence count data. Genome Biol. 11, R106 (2010).
26. Maeder, M. L. et al. Robust, synergistic regulation of human gene expression using TALE activators. Nat Meth 10, 243-245 (2013).
27. Ku, M. et al. Genomewide analysis of PRC1 and PRC2 occupancy identifies two classes of bivalent domains. PLOS Genet. 4, e1000242 (2008).
28. Ram, O. et al. Combinatorial Patterning of Chromatin Regulators Uncovered by Genome-wide Location Analysis in Human Cells. Cell 147, 1628-1639 (2011).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-23. (canceled)

24. A method of reducing methylation of histones associated with a selected DNA sequence in a mammalian cell, the method comprising contacting the cell with a fusion protein comprising an engineered transcription activator-like (TAL) effector repeat array that binds specifically to an enhancer, and
a catalytic domain comprising a sequence that catalyzes histone demethylation.

25. The method of claim 24, wherein the fusion protein further comprises a linker between the engineered TAL effector repeat array and the catalytic domain.

26. The method of claim 24, wherein the catalytic domain comprises full length lysine-specific demethylase 1 (LSD1), or a catalytic domain of LSD1.

27. The method of claim 26, wherein the catalytic domain comprises amino acids 172-833 of SEQ ID NO:1.

28. The method of claim 24, wherein the fusion protein comprises a plurality of catalytic domains, optionally with linkers therebetween.

29. The method of claim 24, wherein the TAL effector repeat array binds specifically to the nucleic acid sequence set forth in SEQ ID NO:6.

30. The method of claim 24, wherein the TAL effector repeat array binds specifically to the nucleic acid sequence set forth in SEQ ID NO:7.

31. The method of claim 24, wherein the TAL effector repeat array binds specifically to the nucleic acid sequence set forth in SEQ ID NO:9.

32. The method of claim 24, wherein the TAL effector repeat array binds specifically to the nucleic acid sequence set forth in SEQ ID NO:10.

33. The method of claim 24, wherein the TAL effector repeat array binds specifically to the nucleic acid sequence set forth in SEQ ID NO:14.

34. The method of claim 24, wherein the TAL effector repeat array binds specifically to the nucleic acid sequence set forth in SEQ ID NO:17.

35. The method of claim 24, wherein the TAL effector repeat array binds specifically to the nucleic acid sequence set forth in SEQ ID NO:19.

36. The method of claim 24, wherein the TAL effector repeat array binds specifically to the nucleic acid sequence set forth in SEQ ID NO:25.

37. The method of claim 24, wherein the TAL effector repeat array binds specifically to the nucleic acid sequence set forth in SEQ ID NO:30.

38. The method of claim 24, wherein the TAL effector repeat array binds specifically to the nucleic acid sequence set forth in SEQ ID NO: 225.
